(19) United States
(12) Patent Application Publication
(10) Pub. No.: US 2010/0247525 A1
(43) Pub. Date: Sep. 30, 2010

(54) COMPOSITIONS AND METHODS FOR TREATING ANTHRAX LETHALITY

(75) Inventors: Edward N. Martin, JR., Charlottesville, VA (US); W. Michael Scheld, Earlysville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/299,952

(22) PCT Filed: May 8, 2007
(86) PCT No.: PCT/US2007/011119

§ 371 Date: Jun. 15, 2010

Related U.S. Application Data
(60) Provisional application No. 60/798,468, filed on May 8, 2006.

(51) Int. Cl. A61K 39/395; A61K 38/02; A61K 31/7088; A61K 31/713; A61P 31/12; A61P 31/04

(52) U.S. Cl. 424/133.1; 514/1.1; 514/44 R; 514/44 A; 424/130.1; 424/142.1; 424/152.1; 424/172.1

(57) ABSTRACT

The present invention provides compositions and methods for preventing and inhibiting anthrax lethality. The present invention relates to protect a subject from anthrax lethality by presensitizing a subject prior to anthrax infection. The present invention further provides compositions and methods for enhancing the innate system to inhibit anthrax-associated lethality. The invention further provides compositions and methods for preventing and inhibiting lethality due to infection regulated via a TNF-α pathway.

### CROSS REFERENCE TO RELATED APPLICATIONS

This application is entitled to priority pursuant to 35 U.S.C. §119(e) to U.S. provisional patent application No. 60/798,468, filed on May 8, 2006, the entirety of which is incorporated by reference herein.

### BACKGROUND

Anthrax is an acute infectious disease caused by the spore-forming bacterium Bacillus anthracis. Anthrax most commonly occurs in wild and domestic lower vertebrates (cattle, sheep, goats, camels, antelopes, and other herbivores). Human infection occurs from direct contact with spores from contaminated animal products (e.g., wool sorter's disease) and typically manifests in one of three forms dependent upon the route of infection: cutaneous, gastrointestinal, and inhalational.

Cutaneous infections occur when the bacterium enters a cut or abrasion on the skin. Deaths are rare with appropriate antimicrobial therapy but cutaneous anthrax infection results in death in approximately 20% of untreated cases.

The gastrointestinal disease form of anthrax may follow the consumption of contaminated meat and is characterized by an acute inflammation of the intestinal tract. Initial signs of nausea, loss of appetite, vomiting, fever are followed by abdominal pain, vomiting of blood, and severe diarrhea. Intestinal anthrax results in death in 25% to 60% of cases.

Inhalational anthrax occurs as a result of inhalation of spores which are then phagocytosed by alveolar macrophages. Once taken up by macrophages, spores germinate into vegetative bacilli and are transported to nearby lymph nodes. From there, the infection often becomes systemic, manifesting as a “flu-like” illness that is rapidly followed by the onset of shock, mediastinal widening, hemoconcentration, hypoalbuminemia, hyponatremia, often meningitis, and death. While antibiotic therapy is usually sufficient to clear bacteria from the host, unless initiated at the onset of infection prior to the accumulation of anthrax toxins, antibiotic therapy does not prevent toxin-associated lethality.

Anthrax is a zoonotic disease that primarily infects animals, and secondarily humans. Although common in less developed parts of the world, anthrax infection remains rare in industrialized countries. Infection occurs from direct contact with spores from contaminated animal products (e.g. wool sorter's disease) and typically manifests in one of three forms dependent upon the route of infection: cutaneous, gastrointestinal, and inhalational. Of these three forms, cutaneous anthrax often responds to antibiotic therapy and is rarely fatal. The gastrointestinal and inhalational forms of the disease are rare under natural circumstances, but are far more severe. Gastrointestinal anthrax is acquired by ingesting food contaminated with Bacillus anthracis and is often fatal if not treated immediately. Inhalational anthrax occurs as a result of inhalation of spores which are then phagocytosed by alveolar macrophages. Once taken up by macrophages, spores germinate into vegetative bacilli and are transported to nearby lymph nodes. From there, the infection often becomes systemic, manifesting as a “flu-like” illness that is rapidly followed by the onset of shock, mediastinal widening, hemoconcentration, hypoalbuminemia, hyponatremia, often meningitis, and death. Antibiotic therapy is usually sufficient to clear bacteria from the host, but unless initiated at the onset of infection prior to the accumulation of anthrax toxins, does not prevent toxin-associated lethality.

Identified pathogens, such as Bacillus anthracis and the various AB toxin-producing bacteria, viruses such as HIV, and illnesses such as “radiation sickness”, induced by gamma and ionizing radiation of lethal or above maximum tolerance exposure, exert their lethal effects by interfering with some portion of the innate immune system, thereby partially paralyzing the innate immunity. The damage to the innate immune system from these insults may range from inactivation of the mitogen-activated phosphorylation kinase (MAPK) pathways, as in anthrax, to damage of catalytic enzymes and destruction of DNA in “radiation sickness”. The common effect of these lethal and damaging agents is to prevent the innate immune system from mounting a robust innate immune defense against the insult.

B. anthracis secretes three exotoxins: protective antigen (PA), lethal factor (LF), and edema factor (EF), which form two bipartite toxins: lethal toxin (LeTx=PA+LF) and edema toxin (EdTx=PA+EF). Protective antigen, an 83 kDa protein (PA83), binds to cell surface receptors where it is cleaved by cell surface-associated proteases into two fragments (63 kDa and 20 kDa)10. The 63 kDa fragment (PA63) remains bound to the cell surface and heptamerizes forming a pore which is then able to facilitate the translocation of EF and LF into the cytosol. EF is an adenylate cyclase which increases intracellular cyclic adenosine monophosphate (cAMP) and has been shown to cause localized edema in vivo. Current literature suggests that EdTx inhibits the innate immune system.

LF is a zinc-dependent metalloprotease that cleaves mitogen-associated kinase kinases (MKKs) 1-4, 6-7, and has been shown to inactivate the p38 and Erk components of the mitogen-associated protein kinase (MAPK) signaling cascade (FIG. 1). LeTx is the primary factor associated with in vivo mortality, although it still remains unclear how cleavage of MKKs directly relates to toxin-associated lethality as there is little correlation between MKK cleavage and cytotoxicity in vitro since MKK cleavage still occurs in cells resistant to LeTx.

There are conflicting observations with some authors citing the BALB/c strain as “sensitive” and others as “resistant” to anthrax lethal toxin (LeTx), and the possibility of endotoxin contamination of earlier LeTx preparations adds yet another layer of uncertainty. Toll-like receptors are an integral part of the innate immune system and recognize relatively conserved pathogen-associated molecular patterns (PAMPs). Ligation of Toll-like receptors (TLR) by bacterial components (e.g. lipoteichoic acid, LTA; lipopolysaccharide, LPS; bacterial lipoprotein, BLP; unmethylated bacterial DNA, CpG DNA; et al.) activates a network of downstream signaling pathways (See FIG. 1), that lead to the synthesis of cytokines as well as acute phase proteins, many of which are integral in determining cell survival in response to bacterial toxins. The same can be said for the Nod1/Nod2 and TLR9 intracellular receptors for peptidoglycan, PGN, and PGN breakdown products, and CPG DNA, respectively.

Over the years, the incredibly high bacteremia observed in anthrax infections (1e8-1e9 cfu/ml) has caused much speculation concerning the role LeTx may play in regulating the innate immune response and facilitating bacterial invasion. One would expect death by septic shock long before this level of systemic bacteremia is reached. A recent paper by Agrawal et al. implicated LeTx-impairment of T-cell priming by dendritic cells as a potential mechanism by which LeTx may impair the adaptive immune system as well as the innate.

In 1979, Galanos et al. demonstrated the ability of D-galactosamine to sensitize rodents, including C57BL/6 mice, to miniscule quantities of LPS22 (Galanos, Proc. Natl. Acad. Sci. 1979; 76:11: 5939-5943). The observable effects and pathways involved in this striking sensitization of mice bears parallels to the rapid anthrax death model we have developed. Moreover, this sensitization by D-galactosamine can be attenuated by pre-sensitization with low-doses of LPS, presumably through the induction of tolerance in macrophages. In this model, it is the inhibition of mRNA and protein synthesis in hepatocytes through the depletion of UTP that leaves the liver vulnerable to normally tolerated levels of LPS and TNF-α. Treatment with LeTx leads to inhibition of Erk1/2 and p38 MAPKs, both of which are required for activation of MAPK-activated protein kinase 1 (MNK1) and cap-dependent protein synthesis (See FIG. 2). LeTx also prevents translocation of NFKB preventing that transcription process generally considered protective from stress events. Inhibition of NFKappaB translocation is an apoptotic correlate in itself. Thus, it is likely that LeTx may sensitize mice to LPS in a manner analogous to D-galactosamine through the impairment of cellular processes.

Despite decades of study, the exact mechanisms involved in LeTx-associated lethality are unknown. Until the last few years, the current model of anthrax pathogenesis in the literature suggested that the high bacteremia associated with anthrax infections led to the development of a septic shock-like syndrome due to high levels of inflammatory cytokines. This hypothesis was replaced by Leppla and Moyeri (J. Clin. Invest. 112:670-682, 2003) with the notion that anthrax toxin lethality was unrelated to inflammatory processes but rather was caused by an undefined mechanism that produced hypoxia and subsequent lethal sequelae. This notion was rapidly accepted as doctrine and remains to this day the current paradigm of anthrax lethal toxin lethality. Thus, productive inquiry into inflammatory events have been largely sidelined as a research paradigm of anthrax lethality.

There is a long felt need in the art for compositions and methods useful for preventing and inhibiting anthrax-associated lethality, particularly LeTx-associated lethality, which is in itself impervious to antibiotic treatment. The present invention satisfies these needs.

### SUMMARY

The present application discloses compositions and methods useful for treating and inhibiting anthrax-associated lethality which is regulated by TNF-α. In addition, the present invention encompasses other infections and responses to toxins which are whose lethality or effects are associated with regulation by TNF-α. The present invention therefore encompasses compositions and methods useful for treating and preventing diseases and disorders by inhibiting TNF-α. The present invention also encompasses additional therapeutic methods for enhancing the innate immune system and presensitizing a subject prior to treatment with an inhibitor of TNF-α.

The present application discloses that pre-administration of small amounts of inflammatory LPS, PGN, repurified LPS, LTA, CpG DNA, or (live or killed) non-toxigenic B. anthracis protects C57BL/6 mice from LeTx-associated mortality. Reversing the procedure, i.e., pre-sensitizing C57bl/6 mice with sub-lethal LeTx followed by sub-lethal amounts of defined inflammatory components (e.g. TLR4 agonist LPS (a surrogate for TLR4 agonist O-anthrolysin) for C57bl/6 and Balb/c mice, TLR2 agonist BLP for Balb/c mice, live or killed B. anthracis or conditioned medium from B. anthracis for either strain) results in 100% death in 3-12 hours. The time to death is dependent on mouse strain and death initiator but always ends in a rapid 100% lethality. This swift lethality is referred to herein as the rapid death paradox. (“RDP”). This RDP model mimics the final stages in human death from anthrax and provides a death mechanism for examination. The principle is simply that the inflammatory component is capable of eliciting a rapid and robust burst of TNF-α, or other inflammatory cytokine, sufficient to initiate the death process and subsequent cytokine/death event cascade. This is analogous to inflammatory hypertoxicity induced by D-galactosamine, here made possible by the anthrax lethal toxin impairment of cellular signal transduction pathways, paralyzing factors downstream of the now inactivated mitogen kinase kinases (MKK) 1-4 and 6, such as NFkappaB, a known survival correlate, and myriad other cellular processes, some of which are given as examples in this report.

In one embodiment, the present invention provides compositions and methods to enhance survival in response to pathogens, toxins, and other agents by stimulating the innate immune system. In one aspect, the diseases encompassed by the invention include influenza, H5N1 and others, TB, and malaria. In one aspect, the pathogens are bacteria. In a further aspect, the pathogen is anthrax. In one embodiment, the innate immune system is enhanced via Toll-Like Receptors. Enhancing agents are all TLR 1-14 and Nod1/Nod2 agonists and include, but are not limited to LPS (TLR 2 and 4), ultra pure LPS (TLR4), PAM-3-CSK-4 (TLR2), PGN (TLR2 and NOD1/NOD2), BLP (bacterial lipoprotein) (TLR 4), MDP (muramyl dipeptide) (TLR2), Lipid A (TLR4), dsRNA (TLR3), poly I:C (TLR3), ssRNA (TLR7), CpG DNA (TLR9), flagellin (TLR 5), and LTA (lipoteichoic acid) (TLR2).

The present invention also encompasses the use of agents as presensitizing agents prior to treatment with a TNF-α inhibiting compound. These agents include the agents listed as enhancers of the innate immune system. Other presensitizing agents include whole killed or live bacteria or sterile culture medium from late log phase bacterial cultures.

The present invention further encompasses the use of antibiotics in conjunction with the use of enhancers, presensitizing agents, and TNF-α inhibiting compounds. The antibiotics include, but are not limited to, ciprofloxacin, clindamycin, doxycycline, merepenem, moxifloxacin, ceftriaxone, amoxicillin, and ampicillin. In one embodiment, at least one antibiotic is administered before the subject is exposed to the pathogen. In one embodiment, the antibiotic is administered after infection, but before the TNF-α inhibiting compound is administered. One of ordinary skill in the art will appreciate that the timing and order of administration of an antibiotic, an enhancer of the innate immune system, presensitizing agents, and a TNF-α inhibiting compound may vary according to the particular situation, such as when or if the subject has already been infected, the extent of the infection, the type of infection, the age or health of the subject, etc.

The present invention provides novel compositions and methods to enhance survival in response to pathogens, toxins, and other agents by modulating the innate immune system. In one aspect, the pathogen is anthrax. In a further aspect, the pathogen may be another bacterium (e.g. Yersinia pestis), a parasite (e.g. Plasmodium sp.), a virus (e.g. influenza) or a venom, toxin, or “radiation” as emanating from nuclear events.

The present invention encompasses administering any single agent, or combination of the following or previously described agents, to enhance survival, attenuate illness, or otherwise provide positive therapy as described above and elsewhere herein. These include, but are not limited to: Lipopolysaccharide (LPS) containing a naturally occurring amount of bacterial lipoprotein (BLP), LPS, highly re-purified to eliminate BLP moieties, BLP, Peptidoglycan (PGN), Lipoteichoic acid (LTA), Zymosan, Mycoplasma lipopeptide (MALP-2), 19 kD fragment of mycobacteria lipopeptide, Lipoarabinomannan, Phosphatidylinositol and the analog PIM and PIM-STF, dsRNA, Unmethylated CpG bacterial DNA, Bacterial flagellin, PHI X 174 virus, monoclonal antibodies to the TLR(1-10) which bind to and stimulate the receptors as biological agonists or antagonists; mAbs; and synthetic peptides or organic constructs which bind to the TLR and act as biological agonists or antagonists as conceived by us for this purpose.

The present invention further encompasses targets of the above-identified agents. For example, enhanced survival or other attenuation of illness in the host is anticipated when the agents listed above are used singly or in some combination against the pathogens, toxins and damaging exposures described herein, which include but are not limited to: Bacillus anthracis or anthrax toxins; Malaria—Plasmodium falciparum (human) or Plasmodium knowlesii (murine); “Radiation sickness” defined above; Vibrio cholera; Yersinia pestis; Corynebacterium diptheriae and diphtheria toxin; Shigella spp. incl. Shigella dysenteria and shiga toxins and shiga-like toxins; Clostridium botulinum and botulinum toxin; Ricin; Staphylococcus spp. incl. S. aureus and the staph enterotoxins; Arboviridae; Filoviridae; Hantaanviridae; and Human immunodeficiency virus.

The present invention also discloses that anthrax lethality, on a background of anthrax lethal toxin cellular impairment, is regulated via inflammatory events initiated by bacterial inflammatory products (e.g. peptidoglycan, bacterial lipoprotein, CpG motif bacterial DNA, RNA, or anthrolysin-O, or other bacterial products not enumerated or any combination of these products) interacting with host cells through receptors or other means, such that the cytokine TNF-α is a frequent or pivotal result of this inflammatory stimulation and an effector or modulator of several ensuing cytokine interactions and effects that affect lethality in the host. Thus, TNF-α is a prominent and rational target for interdiction of host death resulting from anthrax toxin. One of ordinary skill in the art will appreciate that these agents can be administered before other therapeutic agents. Additionally, one of ordinary skill in the art will be able to determine the times and intervals of administration and the doses of each compound to be administered.

In one embodiment, the anthrax lethality pathway is a TNF-α initiated pathway. In another embodiment, the anthrax lethality pathway is a TNF-α regulated pathway. Therefore, the present invention relates to regulating anthrax lethality via regulation of TNF-α, and the downstream events and effects of TNF-α and its signal transduction pathway(s). In one aspect, the present invention provides methods and compositions for preventing anthrax lethality comprising inhibiting TNF-α function. In one aspect, TNF-α is inhibited with an antibody directed against TNF-α. In a further aspect, the antibody is a monoclonal antibody. In one aspect, TNF-α function is inhibited by inhibiting its interaction with its cognate receptor. In another aspect, TNF-α function is inhibited by inhibited function of its receptor. In yet another aspect, TNF-α is inhibited by inhibiting its synthesis and levels and by increasing its degradation.

The present application discloses that human death initiators adoptively transferred to mouse systems are resolved by anti-human TNF-α commercial preparations Etanercept (Enbrel; Amgen and Wyeth) and Infliximab (Remicade; Centocor). Adalimumab (Humira; Abbott Labs) is also encompassed within the present invention. Any other TNF-α attenuator, or attenuator of downstream TNF-initiated cytokines, including, but not limited to IL-1, IL-6, IL-2, IFN, IL-12, M-CSF, IL-15, IL-18, and IL-13, are useful in the invention. Also encompassed within the invention are small molecules that inhibit inflammation such as the adenosine receptor agonists, piceatannol, pirfenidone, colchicine, methotrexate, etc., as well as Toll-like receptor 2 and 4 (TLR2 and TLR4) antagonists or substances that neutralize the inflammation-inducing capacities of bacterial products or components or inactivate the “death domain” (e.g. caspases) of cellular adaptor molecules which are biased by any of the aforementioned inflammatory events or their sequelae.

Based on the disclosure herein describing protective and lethal models (see Examples), the present invention discloses that the temporal activation of signaling pathways by bacterial components is critical in determining survival following challenge with anthrax LeTx. Because of their recognition of conserved pathogen-associated motif patterns (PAMPs), TLRs are encompassed within the invention as mediators of this process. MyD88, an adaptor protein, is shared by the NOD1/NOD2, TLR2, TLR4, and TLR9 pathways and is encompassed by the invention as a downstream target of bacterial inflammatory product activation involved in the protection process.

The present application encompasses the use of siRNA for blocking the pathways identified herein. In one aspect, the siRNA is directed against TNFα or a fragment thereof. In a further aspect, a first siRNA can be used in combination with a second siRNA with a slightly different sequence than the first, or the second siRNA can be directed against a different sequence altogether. An siRNA of the invention can be further used with other regulators described herein, or known in the art, such as peptides, antisense oligonucleotides, nucleic acids encoding peptides described herein, aptamers, antibodies, kinase inhibitors, and drugs/agents/compounds.

The present invention is directed to useful aptamers. In one embodiment, an aptamer is a compound that is selected in vitro to bind preferentially to another compound (in this case the identified proteins). In one aspect, aptamers are nucleic acids or peptides, because random sequences can be readily generated from nucleotides or amino acids (both naturally occurring or synthetically made) in large numbers but of course they need not be limited to these. In another aspect, the nucleic acid aptamers are short strands of DNA that bind protein targets. In one aspect, the aptamers are oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. Transfer of large amounts of a chemically stabilized aptamer into cells can result in specific binding to a polypeptide of interest, thereby blocking its function. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

As used herein, an antagonist or blocking agent may comprise, without limitation, an antibody, an antigen binding portion thereof or a biosynthetic antibody binding site that binds a particular target protein; an antisense molecule that hybridizes in vivo to a nucleic acid encoding a target protein or a regulatory element associated therewith, or a ribozyme, aptamer, or small molecule that binds to and/or inhibits a target protein, or that binds to and/or inhibits, reduces or otherwise modulates expression of nucleic acid encoding a target protein.

Aptamers offer advantages over other oligonucleotide-based approaches that artificially interfere with target gene function due to their ability to bind protein products of these genes with high affinity and specificity. However, RNA aptamers can be limited in their ability to target intracellular proteins since even nuclease-resistant aptamers do not efficiently enter the intracellular compartments. Moreover, attempts at expressing RNA aptamers within mammalian cells through vector-based approaches have been hampered by the presence of additional flanking sequences in expressed RNA aptamers, which may alter their functional conformation.

The idea of using single-stranded nucleic acids (DNA and RNA aptamers) to target protein molecules is based on the ability of short sequences (20 mers to 80 mers) to fold into unique 3D conformations that enable them to bind targeted proteins with high affinity and specificity. RNA aptamers have been expressed successfully inside eukaryotic cells, such as yeast and multicellular organisms, and have been shown to have inhibitory effects on their targeted proteins in the cellular environment.

The present application discloses compositions and methods for inhibiting the proteins described herein, and those not disclosed which are known in the art are encompassed within the invention. For example, various modulators/effectors are known, e.g. antibodies, biologically active nucleic acids, such as antisense molecules, RNAi molecules, or ribozymes, aptamers, peptides or low-molecular weight organic compounds recognizing said polynucleotides or polypeptides.

### BRIEF DESCRIPTION OF THE DRAWINGS

FIG. 1 is a schematic representation of the pathways proposed to be mediated by anthrax. Once in the cytosol, LF cleaves and inactivates members of the MAPK signaling cascade leading to an inhibition in survival pathways and an attenuation of inflammatory cytokines that is most likely the result of inhibition of NFkB activation. EF increases intracellular levels of cAMP, leading to activation of protein kinase A (PKA), inhibition of apoptosis, and NOS synthesis. Inflammatory cell wall components such as peptidoglycan (PGN), lipopolysaccharide (LPS), and lipoteichoic acid (LTA) initiate the synthesis of inflammatory cytokines and chemokines through activation of various transcription factors and translation machinery.

FIG. 2 is a schematic representation of the pathways proposed to be mediated by anthrax. Inflammatory cell wall components such as peptidoglycan (PGN), lipopolysaccharide (LPS), and lipoteichoic acid (LTA) initiate the synthesis of inflammatory cytokines and chemokines through activation of various transcription factors and translation machinery.

FIG. 3 graphically illustrates the ability of cell wall components to protect or induce rapid death mice from LeTx challenge depending on the order of administration. C57BL/6 mice receiving only LeTx (100 μg PA/40 μg LF) exhibited a mortality rate of 90%, whereas those that were pre-sensitized with cell wall components prior to administration of LeTx had a significantly lower mortality rate of 11% (P<0.001). In a reversed order of administration with non-lethal doses, female C57BL/6 mice were pre-sensitized by intraperitoneal (IP) injection of a non-lethal dose (25 μg PA+10 μg LF) of LeTx, or vehicle, 16 hours prior to IP inoculation with a non-lethal dose of LPS (1 mg/kg O111:B4). While all LeTx pre-sensitized mice died by 4 hours (n=36, p<0.001), all mice receiving 1 mg/kg CW alone survived (n=20).

FIG. 4 depicts a graphical illustration of cytokine levels in treated mice. Compared to mice receiving LPS alone, by one hour, LeTx significantly attenuated but did not eliminate the expression of most cytokines from this profile obtained from the serum of C57BL/6 mice. Note this is a log 10 scale.

FIGS. 5-6 document the bacterial products which are death initiators in RDP. Note that only LTA and PGN fail to initiate rapid death on a background of 25/10 LeTx pre-sensitization in C57bl/6 mice. No initiator alone, or LeTx alone, at this dose level is lethal for C57BL/6 mice.

FIGS. 7A and 7B graphically illustrate that killed anthrax BH445 (5×10⁷ CFU) produces rapid death in LeTx 25/10 pre-sensitized C57bl/6 mice. The ordinate indicates percent survival and the abscissa indicates time in hours after killed B. anthracis exposure (closed triangle indicates heat-killed anthrax following presensitization with LeTx; closed circle indicates exposure to heat-killed anthrax only).

FIG. 8 graphically illustrates that a soluble MP component may increase death in BALB/c pseudo-RDP. 2×10⁵ syngeneic MΘs exposed to exposed to 10 μg/ml PGN for 24 hours protect mice from LeTx death. SNs from the same MΘs induced 40% lethality from 100/40 LeTx total n=15. The N for each group was 5. (closed square indicates PGN pretreatment at 2 mg/kg; large closed circle indicates PGN at 10 u/ml MP 0.5 hr and SN at 24 hr; open triangle indicates PGN at 10 u/ml MP 3.0 hr and SN at 24 hr; small closed circle indicates PGN at 10 u/ml MP 8.0 hr and SN at 24 hr; closed triangle indicates 2×105 MP exp 10 μg/ml PGN 24 hr.

FIG. 9 graphically illustrates that TNF-α elevation is a strong positive correlate with lethality in the Rapid Death Paradox model. The ordinate represents TNF-α concentration in pg/ml from mouse serum. Naive C57/BL6 mice as well as the indicated treatments were tested (CpG alone; LeTx+12.5reLPS; PGN alone; LeTx+PGN; reLPS alone; LeTx+reLPS; LTA alone, LeTx+LTA; imLPS; LeTx+imPurLPS).

FIG. 10 graphically illustrates that anti-TNF-α monoclonal antibodies completely inhibit the anthrax rapid death paradox in the BALB/c mouse model. The ordinate indicates percent survivors and the abscissa indicates time in hours following exposure. All four groups receiving an anti-TNF-α had 100% survival (closed diamond indicates mouse TNF-α; closed large square indicates imLPS; closed triangle indicates human TNF-α; large open square indicates B.a. conditioned medium; solid green line indicates anti-mouse TNF-α+mouse TNF-α; second solid green line indicates ant-mouse TNF-α+imLPS; x indicates Remicade+human TNF-α; solid purple line indicates anti-mouse TNF-α+B.a. conditioned medium.

FIG. 11 graphically illustrates that anti-TNF-α monoclonal antibodies completely inhibit the anthrax rapid death paradox in the C57bl/6 mouse model. The ordinate indicates percent survivors and the abscissa indicates time in hours following exposure. Note that human TNF-α was adoptively transferred to the mouse model and initiated RDP via the mouse TNFR1. Death from RDP was subsequently eliminated by neutralizing the human TNF-α death trigger with commercially available and FDA-approved for human use anti-human TNF-α Remicade (Infliximab). The adoptive transfer of human proteins is illustrated by a solid line with a filled triangle. All four groups receiving an anti-TNF-α had 100% survival (closed diamond indicates mouse TNF-α; closed large square indicates imLPS; closed triangle indicates human TNF-α; large open square indicates B.a. conditioned medium; solid green line indicates anti-mouse TNF-α+mouse TNF-α; second solid green line indicates ant-mouse TNF-α+imLPS; x indicates Remicade+human TNF-α; solid purple line indicates anti-mouse TNF-α+B.a. conditioned medium.).

FIG. 12 graphically illustrates that Etanercept prevents lethality in a human TNF-alpha initiated anthrax LeTX pre-administration rapid death paradox model in C57bl/6 mice. The four groups comprise: (closed diamond; 5.0 μg human TNF-α, administered intraperitoneally; N=9) (x; 5.0 μg human TNF-α plus Etanercept administered intraperitoneally; N=5) (closed triangle; 1.0 μg human TNF-α administered intravenously; N=3) (closed circle; 1.0 μg human TNF-α plus Etanercept, administered intravenously; N=5). The ordinate represent percent survival. The abscissa represents time in hours after death initiator human TNF-α.

FIG. 13 graphically illustrates the results of a comparison of the anthrax toxin rapid death paradox (RDP) model in the presence and absence of TNF-α treatment. (closed diamonds—animals pretreated with anthrax lethal toxin followed by challenge with death initiator) (closed triangles—animals pretreated with anthrax lethal toxin followed by challenge with death initiator plus anti-TNF-α). The ordinate represents percent survival. The abscissa represents time in hours after death initiator human TNF-α.

FIG. 14 graphically illustrates the results of a series of treatments demonstrated in bar graph format. FIG. 14 Illustrates TNF-α elevation as a strong positive correlate with lethality in the RDP model. Note that imLPS is both 100% lethal and shows the highest elevation of serum TNF-α concentration when compared to other inflammatory agents that do not produce 100% lethality in this model. The ordinate indicates cytokine concentration in picograms per mL of serum and the abscissa describes experimental variables.

### DETAILED DESCRIPTION OF THE INVENTION

### Abbreviations and Acronyms—

ALO—O-anthrolysin or synonym Anthrolysin-O; a cholesterol-dependent cytolysin which is secreted by B. anthracis and is a potent TLR4 agonist; ALO has been shown to initiate TNF-α release; a proposed synergistic inflammatory death initiator along with other bacterial inflammatory products.
BHI—Brain Heart Infusion bacterial culture medium; used as a growth medium for B. anthracis; not a LeTx death initiator unless “conditioned” by growth of B. anthracis in the medium.
BLP—a cell wall component found in most bacteria; a potent TLR2 agonist and initiator of TNF-α release; Pam3Csk4 is a synthetic BLP analog with the same TNF-α release capability
CFU—colony forming unit
CW—bacterial cell wall
EdTx—edema toxin
EF—edema factor
imLPS—crude commercial preparation of lipopolysaccharide (e.g. LPS 0111:B4; Sigma Chemicals, St. Louis, Mo.) containing BLP and LPS; a potent initiator of TNF-α release; as an agonist of both TLR2 (BLP) and TLR4 (LPS); the Toll-like receptor agonists are possibly synergistic
imPurLPS—another abbreviation for imLPS
LF—lethal factor
LeTx—lethal toxin
LPS—lipopolysaccharide; a bacterial cell wall component and potent inflammatory agent that elicits TNF-α release; ultra pure LPS is a TLR4 agonist while standard preparations are TLR2 and TLR4 agonists due to BLP in the preparation.
LTA—lipoteichoic acid; a Gram+bacterial cell wall component; potent TLR2 agonist and inflammatory agent
LT—anthrax lethal toxin (LeTx)
MAPK—mitogen-activated protein kinase
Mθ—macrophage; a phagocytic immune cell that produces TNF-α
MKK—mitogen associated kinase kinase
MNK1—MAPK-activated protein kinase 1
MP—same as MΘ, i.e. a macrophage
PA—protective antigen the cell-binding entry protein for anthrax lethal factor (LF) and edema factor (EF)
PAMPs—pathogen-associated motif patterns
PGN—peptidoglycan; common bacterial cell wall component; degradation products bind intra-lysosomal receptor NOD1/NOD2; inflammatory substance
RDP—rapid death paradox
reLPS—“repurified” or “ultra pure” LPS containing no BLP and thus an exclusive TLR4 agonist; potent stimulator of TNF-α release
SN—supernatant; typically a “conditioned” cell or bacterial culture medium into which growing cells or bacteria have secreted products
TLR—toll-like receptor
TNF-α—tumor necrosis factor-alpha
TTD—time to death

### Definitions—

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. As used herein, each of the following terms has the meaning associated with it in this section.

The articles “a” and “an” are used herein to refer to one or to more than one (i.e to at least one) of the grammatical object of the article. By way of example, “an element” means one element or more than one element.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

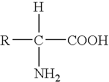

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “anthrax-associated lethality”, as used herein, means mortality due to the effects and sequelae of the toxins released during an anthrax infection. The terms “anthrax-associated lethality” and “anthrax lethality” are used interchangeably herein.

As used herein, “anthrax-associated lethality” regulated by TNF-α refers to mortality caused by an increase in TNF-α or a TNF-α regulated pathway due to an anthrax infection.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

“Antiviral agent,” as used herein means a composition of matter which, when delivered to a cell, is capable of preventing replication of a virus in the cell, preventing infection of the cell by a virus, or reversing a physiological effect of infection of the cell by a virus.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biological sample,” as used herein, refers to samples obtained from a subject, including, but not limited to, skin, hair, tissue, blood, plasma, cells, sweat and urine.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

An “effective amount” or “therapeutically effective amount” of a compound is that amount of compound which is sufficient to produce a selected or desired effect. The term “effective amount” is used interchangeably with “effective concentration” herein.

As used herein, the term “effector domain” refers to a domain capable of directly interacting with an effector molecule, chemical, or structure in the cytoplasm which is capable of regulating a biochemical pathway.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, a “functional” biological molecule is a biological molecule in a form in which it exhibits a property by which it is characterized. A functional enzyme, for example, is one which exhibits the characteristic catalytic activity by which the enzyme is characterized.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

By the term “immunizing a human against an antigen” is meant administering to the human a composition, a protein complex, a DNA encoding a protein complex, an antibody or a DNA encoding an antibody, which elicits an immune response in the human which immune response provides protection to the human against a disease caused by the antigen or an organism which expresses the antigen.

The term “inhibit,” as used herein, refers to the ability of a compound or any agent to reduce or impede a described function. Preferably, inhibition is by at least 10%, more preferably by at least 25%, even more preferably by at least 50%, and most preferably, the function is inhibited by at least 75%.

The term “inhibiting anthrax-associated lethality” means delaying, slowing, or reducing the time to death after anthrax infection.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein, “inhibiting TNF-α” refers to the use of any compound, agent, or mechanism to inhibit TNF-α synthesis, levels, activity, or function are reduced or inhibited as described above.

As used herein, “an inhibitor of TNF-α” refers to any compound, agent, or mechanism whereby TNF-α synthesis, levels, activity, or function are reduced or inhibited as described above.

As used herein, the term “induction of apoptosis” means a process by which a cell is affected in such a way that it begins the process of programmed cell death, which is characterized by the fragmentation of the cell into membrane-bound particles that are subsequently eliminated by the process of phagocytosis.

“Inappropriate apoptosis” of cells refers to apoptosis (i.e. programmed cell death) which occurs in cells of an animal at a rate different from the range of normal rates of apoptosis in cells of the same type in an animal of the same type which is not afflicted with a disease or disorder.

The “innate immune system” as used herein comprises the cells and mechanisms that defend the host from infection by other organisms, in a non-specific manner. This means that the cells of the innate system recognize, and respond to, pathogens in a generic way, but unlike the adaptive immune system, it does not confer long-lasting or protective immunity to the host. Innate immune systems provide immediate defense against infection, and are found in all classes of plant and animal life. By “enhancing the innate immune system” is meant use of any means to stimulate production of inflammatory cytokines leading to induction of a robust Th1 response.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “nucleic acid construct,” as used herein, encompasses DNA and RNA sequences encoding the particular gene or gene fragment desired, whether obtained by genomic or synthetic methods.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with a pathogenic agent. This is sometimes referred to as induction of tolerance.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

The term “regulate” refers to either stimulating or inhibiting a function or activity of interest.

A “receptor” is a compound that specifically binds to a ligand.

A “ligand” is a compound that specifically binds to a target receptor.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, DC, p. 574).

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

“Regulated by TNFα” means affected directly or indirectly by the activity or function of TNFα, and/or by its signal transduction pathway.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of diagnosis or treatment is a mammal, including a human. Non-human animals subject to diagnosis or treatment include, for example, livestock and pets.

The term to “treat,” as used herein, means reducing the frequency with which symptoms are experienced by a patient or subject or administering an agent or compound to reduce the frequency with which symptoms are experienced.

As used herein, “treating a viral disease or disorder” means reducing the frequency with which a symptom of the viral disease or disorder is experienced by a patient. Viral disease or disorder is used interchangeably herein with virus-related disease or disorder and viral-related disease or disorder.

A “therapeutic” treatment is a treatment administered to a subject who exhibits signs of pathology, for the purpose of diminishing or eliminating those signs.

A “prophylactic” treatment is a treatment administered to a subject who does not exhibit signs of a disease or exhibits only early signs of the disease for the purpose of decreasing the risk of developing pathology associated with the disease.

The term “upstream proteins involved in the TNFα protein regulatory pathway” refers to proteins upstream of TNFα which regulate its function, activity, synthesis, levels, etc. The term ‘upstream regulation of the TNFα regulatory pathway” refers to the proteins or other molecules, as well as upstream pathways which regulate TNFα synthesis, levels, activity, function, etc., including the signal transduction pathways regulated by the interaction of TNFα with its cognate receptor.

By the term “vaccine,” as used herein, is meant a composition which when inoculated into an animal has the effect of stimulating an immune response in the animal, which serves to fully or partially protect the animal against a disease or its symptoms. The term vaccine encompasses prophylactic as well as therapeutic vaccines. A combination vaccine is one which combines two or more vaccines.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

A “virus replication-inhibiting amount” as used herein means the amount of an inhibitor necessary to detectably inhibit or reduce virus replication in a cell or an animal, compared with the level of virus replication when the inhibitor is not present. It also means the amount of inhibitor required to reduce virus replication when the inhibitor is added to an animal or cell in which virus replication has already begun, compared to the amount of virus replication in the absence of the inhibitor.

The present invention relates to the present discovery that anthrax lethality can occur via a TNF-α associated pathway. The present application provides compositions and methods for inhibiting anthrax lethality. The present invention further provides compositions and methods for inhibiting TNF-α synthesis, production, levels, and activity, as well as functions regulated by TNF-α.

The present invention provides for inhibiting TNF-α synthesis, processing, activity, levels, and TNF-α-associated pathways using a variety of techniques and reagents. The present invention encompasses any inhibitor of TNF-α, including, but not limited to Remicade (Infliximab) or Enbrel (Etanercept) including the following compounds, drugs and any others not disclosed herein that inhibit the transcription, synthesis, activity, function, binding, processing, or any other inhibition means of TNF-α. A list of known inhibitors of TNF-α is provided below:

| Approved or in Trial | anti-TNF-alpha | |
|---|---|---|
| Name | Compound | Function |
| Infliximab (Remicade) | Chimeric IgG1 anti-TNF-α | Neutralize TNF-α |
| Etanercept (Enbrel) | Hu TNFRp75:Fc IgG1 Fusion Protein | Neutralize TNF-α |
| Adalimumab (Humira) | Human IgG1 anti-TNF-α mAb | Neutralize TNF-α |
| Afelimomab (Segard) | Mouse F(ab′2) anti-TNF-α mAb | Neutralize TNF-α |
| CDP-571 (Humicade) | Humanized mouse anti-TNF-α mAb | Neutralize TNF-α |
| CDP-870 | PEG-hu-F(ab′) anti-TNF fragment | Neutralize TNF-α |
| Onercept | rec sTNFRp55 | Neutralize TNF-α |
| PEG sTNFR1 | PEG rec s TNFR p55 | Neutralize TNF-α |
| CytoTab | Sheep F(ab′) anti-TNF-α | Neutralize TNF-α |
| ISIS-104838 | antisense TNF-α | Inhibit TNF-α transcription |
| | methoxy-oligonucleotide | |
| Pirfenidone | pyridine derivative | Inhibit TNF production via |
| | | NF-kappaB |
| BMS561392 | TACE and MMP Inhibitor | Blocks pre-TNF-α processing |
| Bindarit | benzyl-indazolic derivative | Inhibits TNF-α and CCL2 synthesis |
| Anthrax Edema Toxin, | cAMP elevators | Anti-inflammatory, apoptosis inhibitors |
| adenylate cyclase toxin, | | |
| adenosine A2a receptor | | |
| agonists | | |
| GW3333 | TACE and MMP Inhibitor | Blocks pre-TNF-α processing |
| Kineret | (Anakinra) IL-1 inhibitor | Neutralizes IL-1 |
| Marimastat | TACE and MMP Inhibitor | Blocks pre-TNF-α processing |

In one embodiment, the anti-TNF-α therapy of the invention is useful against the death from anthrax, which occurs after antibiotics have cleared the bacteria from the bloodstream, and therefore in complete treatment of anthrax exposure this therapeutic(s) is an adjunctive therapy that should be administered at the same time as antibiotics. While it is an adjunctive therapy for anthrax bacteremia sequelae, it is the PRIMARY and ONLY existing therapy for anthrax lethal toxin, without which the Bacillus anthracis would be essentially harmless, that has already approved drugs ready for the treatment of deadly anthrax toxemia. The present application encompasses the inhibition by any means of TNF-α, as a new therapeutic of great importance that fills a dire unmet medical and biodefense need.

In one aspect, upregulation of PI3K, SOCS, and IRAK-M can reduce initial TNF-α (see Fukao and Koyasu, 2003, Trends in Immunology, 24:358). The invention therefore encompasses regulators of PI3K, SOCS, and IRAK-M.

Some examples of diseases which may be treated according to the methods of the invention are discussed herein. For example, plague is analogous to anthrax regarding a death signal (see for example, Ruckdeschel et al., 1998, J. Exp. Med., 187:7:1069-79). Therefore, the methods of the present invention are also useful for preventing and treating the plague. The invention should not be construed as being limited solely to these examples, as other TNF-α pathway-associated diseases which are at present unknown, once known, may also be treatable using the methods of the invention. Additionally, other known diseases which have a distinct TNF-α correlate with their pathogenicity also include malarial meningitis, influenzas, hepatitis C and other acute and chronic diseases.

TNF-α can work via apoptotic mechanisms and methods regulating apoptosis are encompassed within the invention. It is known that I-FLICE is an inhibitor of tumor necrosis factor receptor-1 and CD-95-induced apoptosis (Hu, et al., 1997, J. Biol. Chem., 272:28:17255-7). In one aspect, the regulator is a defective caspase. In another aspect, the defective caspase is catalytically inert.

In one embodiment, the invention encompasses isolated nucleic acids. In one aspect, the isolated nucleic acids comprise nucleic acid sequences which encode antibodies or peptides of the invention, or homologs, fragments, derivatives, or modifications thereof. In another aspect, the nucleic acids comprise antisense oligonucleotides.

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89.

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), diinethylene-sulfoxide (—CH₂—SO—CH₂), dimethylene-sulfone (—CH₂—SO₂—CH₂), 2′-O-alkyl, and 2′-deoxy2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

The nucleic acids may be purified by any suitable means, as are well known in the art. For example, the nucleic: acids can be purified by reverse phase or ion exchange HPLC, size exclusion chromatography or gel electrophoresis. Of course, the skilled artisan will recognize that the method of purification will depend in part on the size of the DNA to be purified.

The term nucleic acid also specifically includes nucleic acids composed of bases other than the five biologically occurring bases (adenine, guanine, thymine, cytosine and uracil).

Modified gene sequences, i.e. genes having sequences that differ from the gene sequences encoding the naturally-occurring proteins, are also encompassed by the invention, so long as the modified gene still encodes a protein that functions to stimulate healing in any direct or indirect manner. These modified gene sequences include modifications caused by point mutations, modifications due to the degeneracy of the genetic code or naturally occurring allelic variants, and further modifications that have been introduced by genetic engineering, i.e., by the hand of man.

Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. Changes may be made to increase the activity of a protein, to increase its biological stability or half-life, to change its glycosylation pattern, and the like. All such modifications to the nucleotide sequences encoding such proteins are encompassed by this invention.

Oligonucleotides which contain at least one phosphorothioate modification are known to confer upon the oligonucleotide enhanced resistance to nucleases. Specific examples of modified oligonucleotides include those which contain phosphorothioate, phosphotriester, methyl phosphonate, short chain alkyl or cycloalkyl intersugar linkages, or short chain heteroatomic or heterocyclic intersugar (“backbone”) linkages. In addition, oligonucleotides having morpholino backbone structures (U.S. Pat. No. 5,034,506) or polyamide backbone structures (Nielsen et al., 1991, Science 254: 1497) may also be used.

The examples of oligonucleotide modifications described herein are not exhaustive and it is understood that the invention includes additional modifications of the antisense oligonucleotides of the invention which modifications serve to enhance the therapeutic properties of the antisense oligonucleotide without appreciable alteration of the basic sequence of the antisense oligonucleotide.

In one embodiment, the invention encompasses peptides, proteins, and fragments, homologs, derivatives, and modifications thereof. The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the a-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-,C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used, to monitor purification at each stage in the procedure. Protein purification methods are well known in the art, and are described, for example in Deutscher et al. (ed., 1990, Guide to Protein Purification, Harcourt Brace Jovanovich, San Diego).

It will be appreciated, of course, that the peptides may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for analogs of proteins or peptides encoded by L/STs. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention further encompasses use of the yeast two-hybrid system to identify regulators of the proteins and pathways described herein. Such regulators can be drugs, compounds, peptides, nucleic acids, etc. Such regulators can include endogenous regulators.

Generally, the yeast two-hybrid assay can identify novel protein-protein interactions and compounds that alter those interactions. By using a number of different proteins as potential binding partners, it is possible to detect interactions that were previously uncharacterized. Secondly, the yeast two-hybrid assay can be used to characterize interactions already known to occur. Characterization could include determining which protein domains are responsible for the interaction, by using truncated proteins, or under what conditions interactions take place, by altering the intracellular environment. These assays can also be used to screen modulators of the interactions.

This invention encompasses methods of screening compounds to identify those compounds that act as agonists (stimulate) or antagonists (inhibit) of the protein interactions and pathways described herein. Screening assays for antagonist compound candidates are designed to identify compounds that bind or complex with the peptides described herein, or otherwise interfere with the interaction of the peptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the compound or drug candidate with a peptide identified herein under conditions and for a time sufficient to allow these two components to interact.

In binding assays, the interaction is binding and the complex formed can be isolated or detected in the reaction mixture. In a particular embodiment, one of the peptides of the complexes described herein, or the test compound or drug candidate is immobilized on a solid phase, e.g., on a microtiter plate, by covalent or non-covalent attachments. Non-covalent attachment generally is accomplished by coating the solid surface with a solution of the peptide and drying. Alternatively, an immobilized antibody, e.g., a monoclonal antibody, specific for the peptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with, but does not bind to a particular peptide identified herein, its interaction with that peptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Complete kits for identifying protein-protein interactions between two specific proteins using the two-hybrid technique are available. This system can also be extended to map protein domains involved in specific protein interactions as well as to pinpoint amino acid residues that are crucial for these interactions.

Compounds that interfere with the interaction of a peptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the peptide may be added to a cell along with the compound to be screened for a particular activity and the ability of the compound to inhibit the activity of interest in the presence of the peptide indicates that the compound is an antagonist to the peptide. The peptide can be labeled, such as by radioactivity.

Other assays and libraries are encompassed within the invention, such as the use of phylomers® and reverse yeast two-hybrid assays (see Watt, 2006, Nature Biotechnology, 24:177; Watt, U.S. Pat. No. 6,994,982; Watt, U.S. Pat. Pub. No. 2005/0287580; Watt, U.S. Pat. No. 6,510,495; Barr et al., 2004, J. Biol. Chem., 279:41:43178-43189; the contents of each of these publications is hereby incorporated by reference herein in their entirety). Phylomers® are derived from sub domains of natural proteins, which makes them potentially more stable than conventional short random peptides. Phylomers® are sourced from biological genomes that are not human in origin. This feature significantly enhances the potency associated with Phylomers® against human protein targets. Phylogica's current Phylomer® library has a complexity of 50 million clones, which is comparable with the numerical complexity of random peptide or antibody Fab fragment libraries. An Interacting Peptide Library, consisting of 63 million peptides fused to the B42 activation domain, can be used to isolate peptides capable of binding to a target protein in a forward yeast two hybrid screen. The second is a Blocking Peptide Library made up of over 2 million peptides that can be used to screen for peptides capable of disrupting a specific protein interaction using the reverse two-hybrid system.

The Phylomer® library consists of protein fragments, which have been sourced from a diverse range of bacterial genomes. The libraries are highly enriched for stable subdomains (15-50 amino acids long). This technology can be integrated with high throughput screening techniques such as phage display and reverse yeast two-hybrid traps.

In one aspect of the invention, the inhibitor of TNF-α is an antibody. The antibody can be an antibody that is known in the art or it can be an antibody prepared using known techniques and the published sequence of TNF-α. The antibody may also be one which is prepared against any of the precursors of TNF-α or against molecules which regulate TNF-α synthesis upstream from TNF-α. The antibody may also be directed against a protein downstream from TNF-α in it signal transduction pathway, such as its cognate receptor.

In one aspect, the antibody is selected from the group consisting of a polyclonal antibody, a monoclonal antibody, a humanized antibody, a chimeric antibody, and a synthetic antibody.

The invention includes a method by which an antibody inhibitor can be generated and used as an inhibitor of TNF-α synthesis or function. Antibodies can be prepared against TNF-α or other proteins of the pathway of TNF-α synthesis or against other molecules which are part of the pathway. The preparation and use of antibodies to inhibit protein synthesis or function or to inhibit other molecules or their synthesis is well known to those skilled in the art, and is described for example in Harlow et al. (Harlow et al., 1988, Antibodies: A Laboratory Manual, Cold Spring Harbor, New York; Harlow et al., 1999, Using Antibodies: A Laboratory Manual, Cold Spring Harbor Laboratory Press, NY). Antibodies of the invention can also be used to detect proteins or other molecules which may be components of the TNF pathway.

The generation of polyclonal antibodies is accomplished by inoculating the desired animal with the antigen and isolating antibodies which specifically bind the antigen therefrom.

Monoclonal antibodies can be used effectively intracellularly to avoid uptake problems by cloning the gene and then transfecting the gene encoding the antibody. Such a nucleic acid encoding the monoclonal antibody gene obtained using the procedures described herein may be cloned and sequenced using technology which is available in the art.

Monoclonal antibodies directed against full length or peptide fragments of a protein or peptide may be prepared using any well known monoclonal antibody preparation procedure. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide or other molecules are generated from mice immunized with the peptide using standard procedures as referenced herein. A nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the existing technology described in, for example, Wright et al., id., and in the references cited therein, and in Gu et al. (1997, Thrombosis and Hematocyst 77:755-759), and other methods of humanizing antibodies well-known in the art or to be developed.

Techniques are also well known in the art which allow such an antibody to be modified to remain in the cell. The invention encompasses administering a nucleic acid encoding the antibody, wherein the molecule further comprises an intracellular retention sequence. Such antibodies, frequently referred to as “intrabodies”, are well known in the art and are described in, for example, Marasco et al. (U.S. Pat. No. 6,004,490) and Beerli et al. (1996, Breast Cancer Research and Treatment 38:11-17).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, NY).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In one embodiment, the antibodies are made against TNF-α, or against homologs, derivatives, or fragments thereof. In another aspect of the invention, antibodies can be made against other components of the TNF-α pathway. Such an antibody may be prepared to bind and inhibit function or to inhibit binding with its cognate receptor.

Inhibiting TNF-α Synthesis, Production, Accumulation and Function Using Antisense Techniques

In one embodiment, antisense nucleic acids complementary to TNF-α mRNA can be used to block the expression or translation of the corresponding mRNA. Antisense oligonucleotides as well as expression vectors comprising antisense nucleic acids complementary to nucleic acids encoding a TNF-α can be prepared and used based on techniques routinely performed by those of skill in the art, and described, for example, in Sambrook et al. (1989, Molecular Cloning: A Laboratory Manual, Cold Spring Harbor Laboratory, New York), in Ausubel et al. (1997, Current Protocols in Molecular Biology, John Wiley & Sons, New York), and in Gerhardt et al. (eds., 1994, Methods for General and Molecular Bacteriology, American Society for Microbiology, Washington, DC). The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497). Oligonucleotides which contain at least one phosphorothioate modification are known to confer upon the oligonucleotide enhanced resistance to nucleases. Specific examples of modified oligonucleotides include those which contain phosphorothioate, phosphotriester, methyl phosphonate, short chain alkyl or cycloalkyl intersugar linkages, or short chain heteroatomic or heterocyclic intersugar (“backbone”) linkages. In addition, oligonucleotides having morpholino backbone structures (U.S. Pat. No. 5,034,506) or polyamide backbone structures (Nielsen et al., 1991, Science 254: 1497) may also be used.

The examples of oligonucleotide modifications described herein are not exhaustive and it is understood that the invention includes additional modifications of the antisense oligonucleotides of the invention which modifications serve to enhance the therapeutic properties of the antisense oligonucleotide without appreciable alteration of the basic sequence of the antisense oligonucleotide.

Phosphorothioate oligonucleotides, which have very low sensitivity to nuclease degradation, may be used. Some oligonucleotides may be prepared lacking CG motifs, which should help reduce toxicity for in vivo use.

In another aspect, antisense nucleic acids complementary to TNF-α mRNAs, can be used to block TNF-α synthesis, and subsequently TNF-α function and stimulated pathways. This can be done by transfecting an appropriate antisense sequence. Antisense nucleic acids may be readily prepared using techniques known to those skilled in the art.

The antisense oligonucleotide inhibitors of TNF-α may be used independently in the cell culture systems or administered to animals. In one embodiment of the invention, the inhibitor of TNF-α is an oligonucleotide, preferably from 5 to 25 nucleotides in length. In another embodiment, the oligonucleotide is from 25 to 50 nucleotides in length. In yet another embodiment, the oligonucleotide is from 50 to 100 nucleotides in length. In a further embodiment, the oligonucleotide is 100-400 nucleotides in length.

Phosphorothioate oligonucleotides enter cells readily without the need for transfection or electroporation, which avoids subjecting the cells to nonspecific inducers of a stress response that might confound the experiment. The oligonucleotides may be administered using several techniques known to those of skill in the art and described herein. Effective inhibitory concentrations for phosphorothioates range between 1 and 50 μN, so a titration curve for diminution of TNF-α signal in western blots can be done to establish effective concentrations for each oligonucleotide used. Once inside the cells, the phosphorothioate-oligonucleotides hybridize with the nascent mRNA very close to the transcriptional start site, a site having maximum effect for antisense oligonucleotide inhibition.

The ability to selectively inhibit transcription of TNF-α or other genes with specific antisense molecules is expected to also allow the inhibition of induction of TNF-α synthesis in the diseases, disorders and conditions described herein, such as anthrax. Thus, the invention provides methods for the use of antisense oligonucleotides that will be effective at diminishing steady-state levels of the protein of interest.

The invention should not be construed to include only TNF-α inhibition using antisense techniques, but should also be construed to include inhibition of other genes and their proteins which are involved in the TNF-α pathway. Furthermore, the invention should not be construed to include only these particular antisense methods described herein.

Using Compounds to Inhibit TNF-α Synthesis

In one embodiment the invention includes a method of inhibiting TNF-α synthesis in a mammal, said method comprising administering to a mammal an effective amount of an inhibitor of TNF-α synthesis, or a derivative or modification thereof, thereby inhibiting TNF-α synthesis in a mammal. Preferably, the mammal is a human.

In one embodiment, the inhibitor comprises from about 0.0001% to about 15% by weight of the pharmaceutical composition. In one aspect, the inhibitor is administered as a controlled-release formulation.

Compounds and Methods Useful for Inhibiting TNF-α Function

The invention, as disclosed herein, relates to the involvement of TNF-α as a key regulator in the lethality resulting from anthrax infection. The invention further relates to methods of inhibiting the function of TNF-α in order to alleviate or treat anthrax-associated symptoms. The invention also relates to the involvement of TNF-α in other diseases and disorders. Inhibition of TNF-α function can be direct or indirect. Therefore, TNF-α function may be inhibited or caused to decrease using many approaches as described herein Inhibition of TNF-α function may be assayed or monitored using techniques described herein as well as others known to those of skill in the art. Function can be measured directly or it can be estimated using techniques to measure parameters which are known to be correlative of TNF-α function. The invention should also be construed to include the use of compounds to modulate other TNF-α functions as well.

In one embodiment, the inhibitor comprises from about 0.0001% to about 15% by weight of the pharmaceutical composition.

The invention should be construed to include various methods of administration, including intravenous, intraperitoneal, topical, oral, intramuscular, intrathecal, vaginal, rectal, subcutaneous, and buccal. The route(s) of administration will be readily apparent to the skilled artisan and will depend upon any number of factors including the type and severity of the disease being treated, the type and age of the veterinary or human subject being treated, and the like.

By way of example, an inhibitor of TNF-α function may be an isolated nucleic acid encoding a nucleic acid sequence which is complementary to a TNF-α mRNA and in an antisense orientation. Other inhibitors include an antisense oligonucleotide, an antibody, or other compounds or agents such as small molecules.

It should be understood that compositions and methods for inhibiting pathways, events, and precursors leading to the synthesis or production of TNF-α, may inhibit not only TNF-α synthesis, but also its accumulation, and ultimately its function. The invention should be construed to include compositions and methods to inhibit all pathways and precursors leading to TNF-α synthesis.

The invention provides methods for identifying inhibitors of anthrax lethality by identifying inhibitors of TNF-α. In general, methods for the identification of a compound which effects the synthesis, production, accumulation or function of TNF-α, include the following general steps:

The test compound is administered to a cell, tissue, sample, or subject, in which the measurements are to be taken. A control is a cell, tissue, sample, or subject in which the test compound has not been added. A higher or lower level of the indicator or parameter being tested, i.e., TNF-α levels, synthesis, function, degradation, etc., in the presence of the test compound, compared with the levels of the indicator or parameter in the sample which was not treated with the test compound, is an indication that the test compound has an effect on the indicator or parameter being measured, and as such, is a candidate for inhibition of the desired activity. Test compounds may be added at varying doses and frequencies to determine the effective amount of the compound which should be used and effective intervals in which it should be administered. In another aspect, a derivative or modification of the test compound may be used.

The invention relates to the administration of an identified compound in a pharmaceutical composition to practice the methods of the invention, the composition comprising the compound or an appropriate analog, homolog, derivative, modification, or fragment of the compound and a pharmaceutically-acceptable carrier. For example, a chemical composition with which an appropriate inhibitor of enzyme dependent production of TNF-α, or inhibitor of TNF-α accumulation or function, or stimulator of TNF-α removal, or degradation, is combined, is used to administer the appropriate compound to an animal. The invention should be construed to include the use of one, or simultaneous use of more than one, inhibitor of TNF-α or stimulator of TNF-α removal, and degradation. When more than one stimulator or inhibitor is used, they can be administered together or they can be administered separately.

In one embodiment, the pharmaceutical compositions useful for practicing the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 100 g/kg/day.

Pharmaceutically acceptable carriers which are useful include, but are not limited to, glycerol, water, saline, ethanol, and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Controlled- or sustained-release formulations of a pharmaceutical composition of the invention may be made using conventional technology.

The source of active compound to be formulated will generally depend upon the particular form of the compound. Small organic molecules and peptidyl or oligo fragments can be chemically synthesized and provided in a pure form suitable for pharmaceutical/cosmetic usage. Products of natural extracts can be purified according to techniques known in the art. Recombinant sources of compounds are also available to those of ordinary skill in the art.

Liquid derivatives and natural extracts made directly from biological sources may be employed in the compositions of this invention in a concentration (w/v) from about 1 to about 99%. Fractions of natural extracts and protease inhibitors may have a different preferred rage, from about 0.01% to about 20% and, more preferably, from about 1% to about 10% of the composition. Of course, mixtures of the active agents of this invention may be combined and used together in the same formulation, or in serial applications of different formulations.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to a subject as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the subject, etc.

It will be recognized by one of skill in the art that the various embodiments of the invention as described above relating to methods of preventing or treating anthrax lethality and inhibiting TNF-α or treating TNF-α pathway regulated diseases or conditions, includes other diseases and conditions not described herein.

The compounds of the invention may be administered to, for example, a cell, a tissue, or a subject by any of several methods described herein and by others which are known to those of skill in the art.

Kits

The present invention should be construed to include kits for treating, preventing, or inhibiting anthrax lethality and for inhibiting or stimulating TNF-α, treating TNF-α associated diseases and disorders, kits for measuring TNF-α and TNF-α related parameters. The invention includes a kit comprising an inhibitor of anthrax lethality or a compound identified in the invention, a standard, and an instructional material which describes administering the inhibitor or a composition comprising the inhibitor or compound to a cell or an animal. In one aspect, the inhibitor is an inhibitor of TNF-α. This should be construed to include other embodiments of kits that are known to those skilled in the art, such as a kit comprising a standard and a (preferably sterile) solvent suitable for dissolving or suspending the composition of the invention prior to administering the compound to a cell or an animal. Preferably the animal is a mammal. More preferably, the mammal is a human.

In accordance with the present invention, as described above or as discussed in the Examples below, there can be employed conventional clinical, chemical, cellular, histochemical, biochemical, molecular biology, microbiology and recombinant DNA techniques which are known to those of skill in the art. Such techniques are explained fully in the literature. See for example, Sambrook et al., 1989 Molecular Cloning—a Laboratory Manual, Cold Spring Harbor Press; Glover, (1985) DNA Cloning: a Practical Approach; Gait, (1984) Oligonucleotide Synthesis; Harlow et al., 1988 Antibodies—a Laboratory Manual, Cold Spring Harbor Press; Roe et al., 1996 DNA Isolation and Sequencing: Essential Techniques, John Wiley; and Ausubel et al., 1995 Current Protocols in Molecular Biology, Greene Publishing.

The invention is now described with reference to the following Examples. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### Examples

### Example 1

Pre-sensitization of C57BL/6 mice with inflammatory cell wall components reduces anthrax lethal toxin induced mortality. Induction of “tolerance” limits or eliminates subsequent inflammatory response from inflammatory stimuli.

Early literature suggested that 12.5 μg of anthrax PA injected simultaneously with 2.5 μg of anthrax LF would produce 80% to 100% mortality in BALB/c mice within five days, mimicking most of the lethal effects of toxic Bacillus anthracis infection. However, we soon learned that this dose did not produce the desired effects in BALB/c or C57BL/6 mice, so we performed susceptibility studies in 10 rodent strains to identify a model system that closely mimicked anthrax infection in humans.

It is presently disclosed that doses of LeTx (recombinant toxins from List Biological Laboratories, CA) required to produce this effect were generally much larger than published results and that the actual mouse strain susceptibility often differed from that reported in the literature. Because of its reliable response to LeTx and its strong cell-mediated immune response (Th1), we selected the C57BL/6 as our preferred model. Later, we identified the BALB/c mouse strain to be a model of interest as a counterpoint to the C57BL/6 strain. BALB/c mice exhibit a weaker cell-mediated immune response than the C57BL/6 and a much stronger humoral response (Th2). In the studies described herein, BALB/c mice are consistently more resistant to LeTx than C57BL/6, although Balb/c macrophages are more sensitive to LeTx-induced apoptosis. It should be noted that many studies of mouse strain “susceptibility” to LeTx were based on extrapolations from susceptibility observed with mouse macrophages from these same strains, in vitro. The present data suggest that much of the extrapolation in the art is erroneous.

Several years ago, then current literature suggested that macrophage cytolysis played an important role in triggering shock and subsequent death in LeTx models. Later, it was reported that pre-sensitization of C57BL/6 peritoneal macrophages with cell wall components could sensitize these normally “resistant” cells to LeTx-induced killing. Because of these published observations, it was reasoned in the present studies that in vivo pre-sensitization of C57BL/6 mice with inflammatory cell wall components (CW) would produce an enhanced toxin effect by stimulating macrophages, resulting in a higher mortality rate and a shorter time to death (TTD) in the C57BL/6 mouse model. Surprisingly, rather than enhancing mortality, pre-sensitization of mice with sub-lethal doses of PGN, LTA or LPS provided protection from the deleterious effects of LeTx (Table 1).

TABLE 1
Mortality and time to death for C57bl/6 from toxins.

| | | | |
|---|---|---|---|
| N = | # Dead | Mortality % | Mean TTD (Hr) |

LeTx Alone (100 μgPA/40 μgLF)

| | | | |
|---|---|---|---|
| 40 | 32 | 90 | 99 |

Cell Wall (LPS, PGN or LTA) Followed By LeTx (100 μgPA/40 μgLF)

| | | | |
|---|---|---|---|
| 40 | 4 | 10 | 127 |

### Example 2

A Rapid Death Paradox is Observed in Anthrax Lethal Toxin (LeTx) Pre-Sensitized C57BL/6 Mice After Inoculation with a Non-lethal Dose of Inflammatory Bacterial Product, e.g. LPS, reLPS or CpG DNA

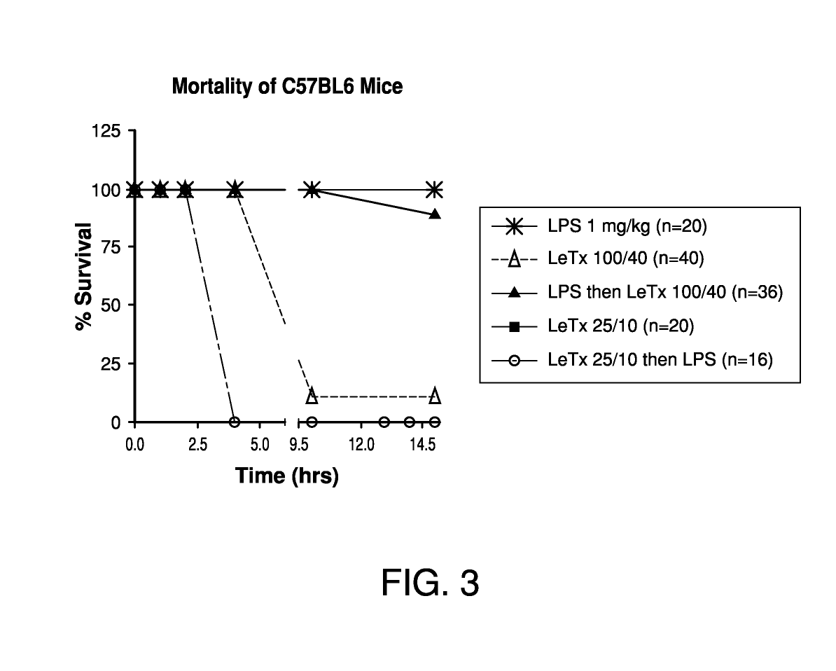

Because data disclosed herein demonstrated that pre-sensitizing C57BL/6 mice with inflammatory cell wall components demonstrated such striking results in producing a large reduction in LeTx-associated mortality, it was further reasoned that the mechanisms of inflammation and anthrax toxin lethal effects at least intersected. For this reason, the two events were then addressed as interrelated rather than separate processes. To that end, the order of pre-sensitization was reversed, using non-lethal doses of bacterial component after pre-sensitization with small, non-lethal amounts of LeTx, and were quite surprised with the results. With the reversed protocol, LeTx first, we observed 100% morality within 3 hours of administration of cell wall component, LPS (FIG. 3). This suggests that the mortality associated with LeTx may occur indirectly, and that the final death blow could actually be triggered by normally tolerated inflammatory stimuli.

It is recognized that B. anthracis does not contain LPS; however, B. anthracis does contain O-anthrolysin (ALO) which is a TLR4 agonist, BLP, a TLR2 agonist, PGN, a Nod1/Nod2agonist and CpG DNA, a TLR9 agonist, any of which may well serve as a “death switch” in our RDP model alone or together, synergistically or individually producing the requisite concentration of TNF-α initiating death. Impure commercial preparations of LPS (e.g., Sigma, O111:B4 and Sigma O26:B6 LPS), both a TLR2 and TLR4 agonist, repurified LPS (TLR4 agonist) and BLP (Pam3Csk4 synthetic BLP; a pure TLR2 agonist) are death initiators. Furthermore, in the RDP model, bacterial growth-conditioned BHI (brain-heart-infusion broth) broth in which 1E8 BH445 non-toxigenic Bacillus anthracis were grown initiates the RDP as readily as LPS. The conditioned broth is purified of bacteria by centrifugation, removal of the top half of the SN and sterile-filtering the SN through a 0.2 μm Millipore filter. Also, the bacterial pellet initiates RDP. The RDP is directly initiated by bacteria whether they are alive, heat-killed, antibiotic-killed, or ethanol-killed, precluding some artifact from aggregation due to the manner of bacterial killing. Finally, the synthetic bacterial lipoprotein (BLP) named PAM₃-CSK-4 (Invivogen, San Diego, Calif.), a selective TLR2 agonist initiates RDP in Balb/c mice but not in C57bl/6 mice at twice the dose level.

From these two models, one protective and one lethal, it is clear that the temporal activation of signaling pathways by bacterial components is critical in determining survival following challenge with anthrax LeTx. Because of their recognition of conserved pathogen-associated motif patterns (PAMPs), TLRs are a good candidate to mediate this process. MyD88, an adaptor protein, is shared by the NOD1/NOD2, TLR2, TLR4, and TLR9 pathways and would seem to be an ideal candidate as the downstream target of bacterial inflammatory product activation involved in the protection process.

### Example 3

LeTx Attenuates Cytokines and Impairs the Innate Immune System

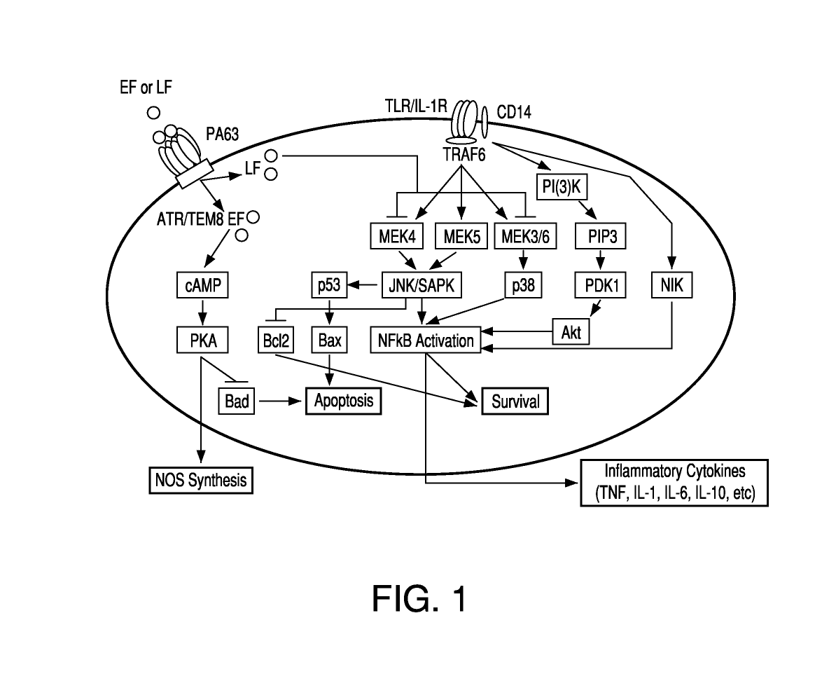

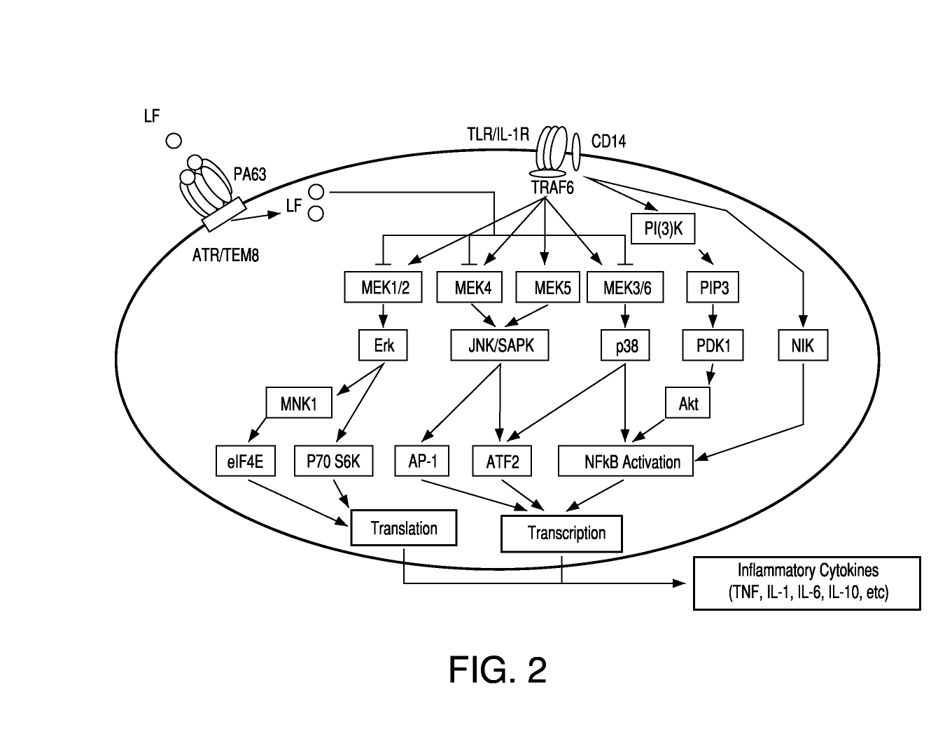

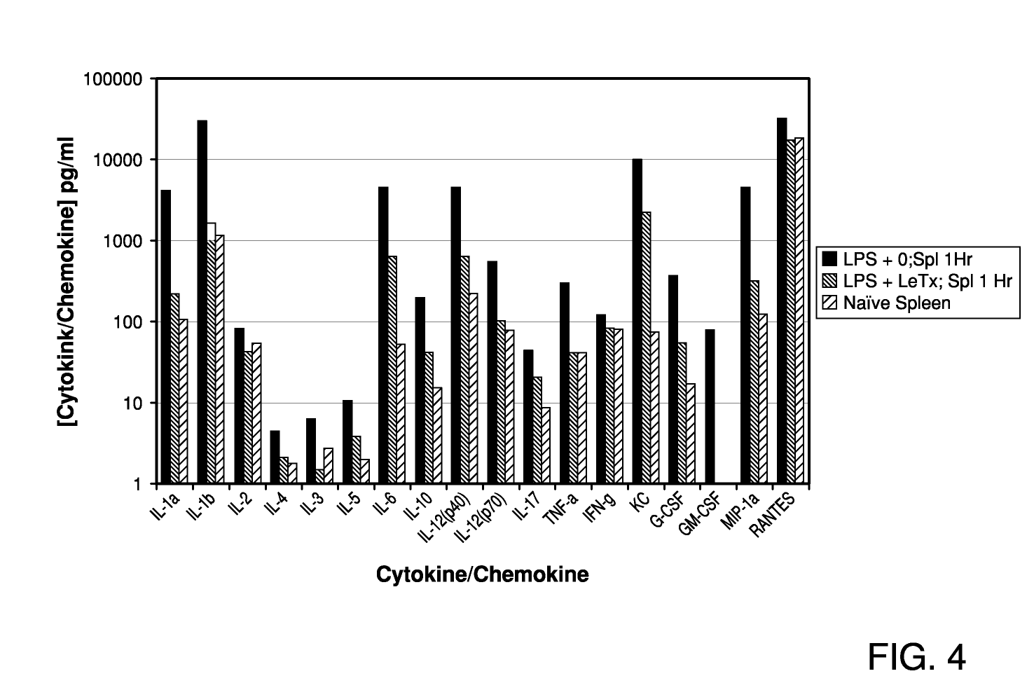

In this setting, a non-lethal dose of LeTx attenuates, but does not eliminate nearly all inflammatory chemokines and cytokines (FIG. 4) as elicited by inflammatory bacterial components. The near-global attenuation of cytokines suggests that LeTx inhibits a central pathway required for the induction of cytokine and chemokine production in vivo. NFkB is a good candidate for this central pathway mediator, as activation of p38 or Erk leads to activation of NFkB in response to TLR ligation. Because these pathways represent two of the prime targets of LeTx in vitro, it is reasonable to suspect that NFkB activation may be impaired by LeTx-inactivation of the MAPK cascade. Alternatively, cytokine and chemokine attenuation could occur through the regulation of transcription factors (e.g. NFkB, ATF2, AP-1, etc.) or translational machinery (e.g. eIF5A, eIF4E) by LeTx (See FIG. 1).

Pre-Sensitizing C57BL/6 Mice with Anthrax Lethal Toxin (LeTx) Inhibits Leukocyte Extravasation Following LPS Challenge.

Based on the results showing the marked attenuation of inflammatory cytokines in vivo, and the data that LeTx impairs the innate immune response, blood profiles were measured using a Hemavet® 850 differential cell counter. Inoculation of C57BL/6 mice with inflammatory bacterial products produces an acute phase inflammatory reaction mediated by cytokines that leads to ordered extravasation of leukocytes (WBC) from the peripheral vasculature into the challenged tissue, a process characterized by a decrease in peripheral blood WBC concentrations. Elevated numbers of circulating WBC during the acute phase of inflammation can indicate unactivated adhesion events resulting in limited trafficking of effector cells to extravascular inflammatory sites. Pre-sensitization of C57BL/6 mice with LeTx leads to increased peripheral WBC concentrations which we hypothesize is due to decreased extravasation into challenged tissues. This suggests that LeTx impairment of expected normal extravasation in response to inflammatory stimuli probably occurs through the inhibition of cytokines, chemokines and adherence factors. This observed and documented phenomenon indicates a broad dismantling of normal cellular processes by LeTx, which then sets the stage for rapid (massive apoptotic) death and subsequent organ system failure, initiated by normally tolerated inflammatory signals.

### Example 4

Survival Enhancement is a new Role for Anthrax EdTx as it Prevents Mortality from Post-exposure Anthrax Lethal Toxemia (LeTx). Modulation of immunity is a general strategy of B. anthracis proliferation. EdTx is a cAMP upregulator. cAMP and the resulting nitric oxide (NO) are recognized inhibitors of apoptosis and platelet activation as well as vasodilators.

Anthrax EdTx protects the ultra sensitive Fisher 344 rat from anthrax lethal toxin mortality. We studied survival in the Fisher 344 rat after injection of an ˜4× LD₁₀₀ dose of anthrax LeTx, 100 micrograms PA/40 micrograms LF (100/40 LeTx), with administration of 5 micrograms PA/2.5 micrograms EF (5/2.5 EdTx) via the tail vein with EdTx injection repeated twice more at 4 hour intervals. Fisher 344 survival from 100/40 LeTx (n=60) is 0%. By administering EdTx with LeTx, we realized a survival rate of 75% (p<0.009) after 3 days even when the toxins were given with a massive 5E8 CFU of non-toxigenic anthrax bacteria (n=8). Note that the EdTx-treated rats did not begin to die until well after cessation of EdTx treatment. This suggests EdTx adjunctive therapy may be prolonged (as with antibiotic therapy) to effectively prevent LeTx-associated mortality. This dramatic result has never been accomplished in an end point post-exposure model of anthrax toxemia. We have shown similar efficacy in our unique models of anthrax death in C57BL/6 and BALB/c mice (data not shown). We have also used EdTx to completely protect C57bl/6 mice from lethal blood-stage murine malaria (data not shown).

The results disclosed herein strongly suggest that death from LeTx is the result of an indirect process in which the host becomes unable to cope with normally tolerated, even beneficial, levels of inflammation following LeTx suppression of critical transcriptional survival pathways. This suggests for the first time that an intricate interplay exists between anthrax toxins, bacteremia, inflammation, and host survival pathways in determining the final but delayed outcome of anthrax infections. As a cAMP enhancer, EdTx is known to attenuate apoptosis, TNF-α production and release, as well as to modulate other immune functions. This anthrax strategy of host immune system manipulation allows a many-fold increase in spore generating bacteria to proliferate , without prematurely killing the host, resulting in an incredible titer of nearly indestructible spores to perpetuate the Bacillus anthracis future survival.

### Example 5

Innate Immunity Modulation by Inflammatory Factors through Signal Transduction Pathways can be Protective or Death Initiators.

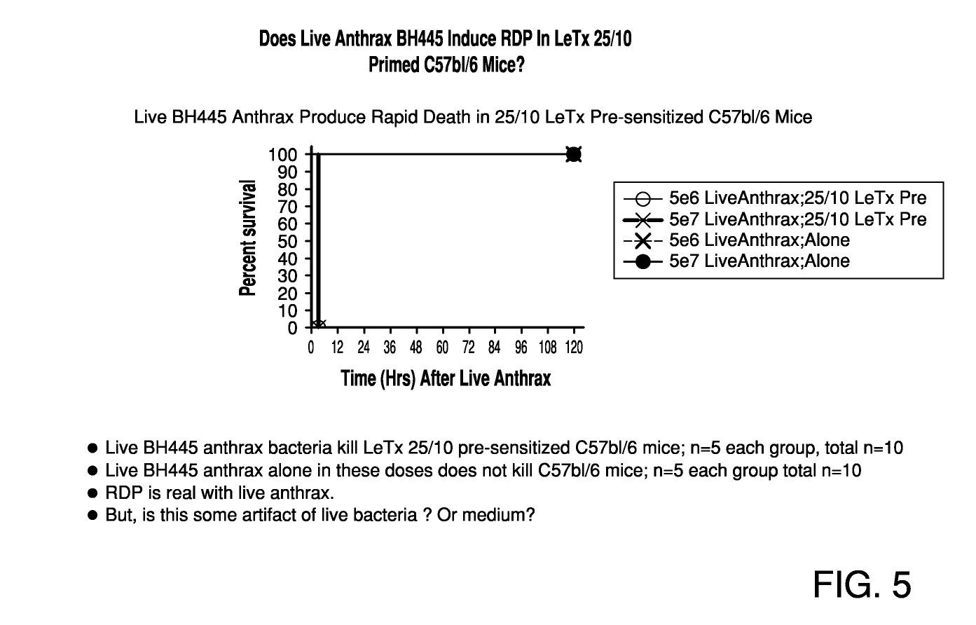

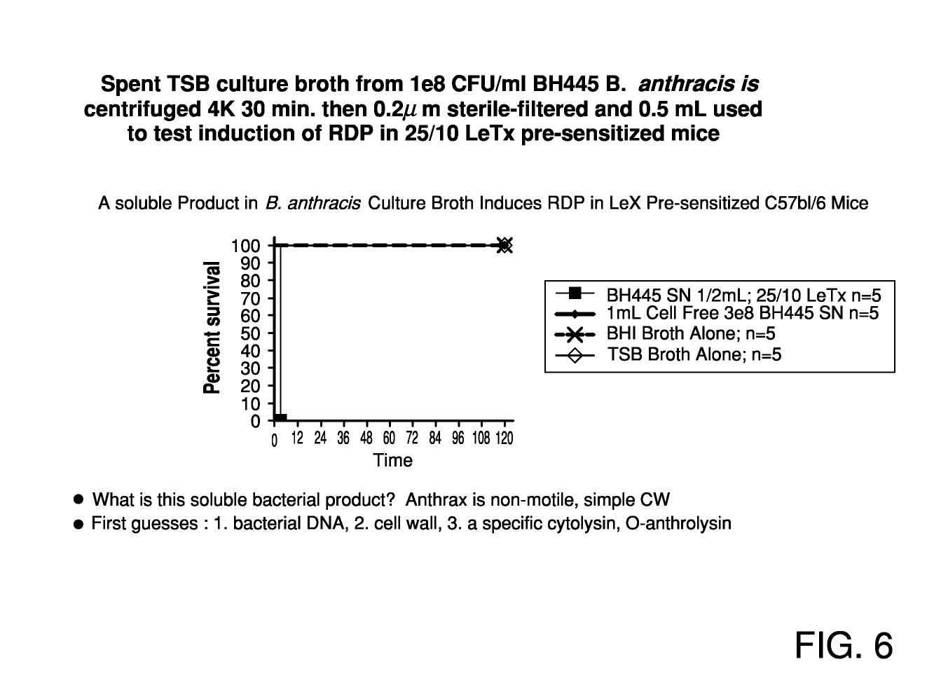

Drawing on the disclosed unique ‘rapid death paradox’ models mimicking the late stage ‘sudden death’ seen in humans, the initiators of death as found in the Bacillus anthracis milieu were investigated. Data from some of these studies is summarized below (see FIGS. 5-6).

The RDP results presented in brevis above are paradigm shifting. Condensed observations concerning the effect of signal transducing receptors on the ‘rapid death paradox’, RDP, in C57BL/6 and BALB/c mice indicate that signal transduction by the Toll-like receptors or any other means sufficient to raise the inflammatory cytokine levels, in particular TNF-α, above a threshold as yet imprecisely defined, or to induce via signal transduction, or other means, any necrotic or apoptotic cascade, any caspase activation, mitochondrial dysfunction resulting in membrane potential alteration or release of cytochrome C into the cytosol, or activation of one of the several of the known “death domains” (e.g. TNF-α or FADD) is sufficient to initiate the Rapid Death Paradox, leading rapidly to systemic organ system dysfunction and death.

### Example 6

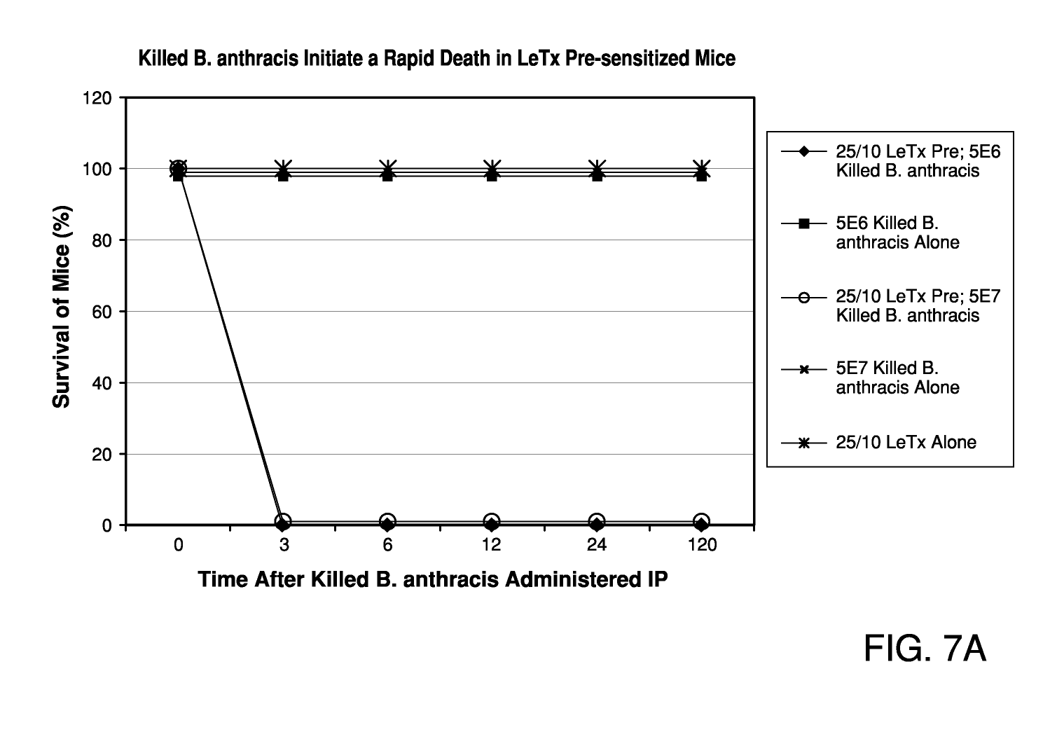

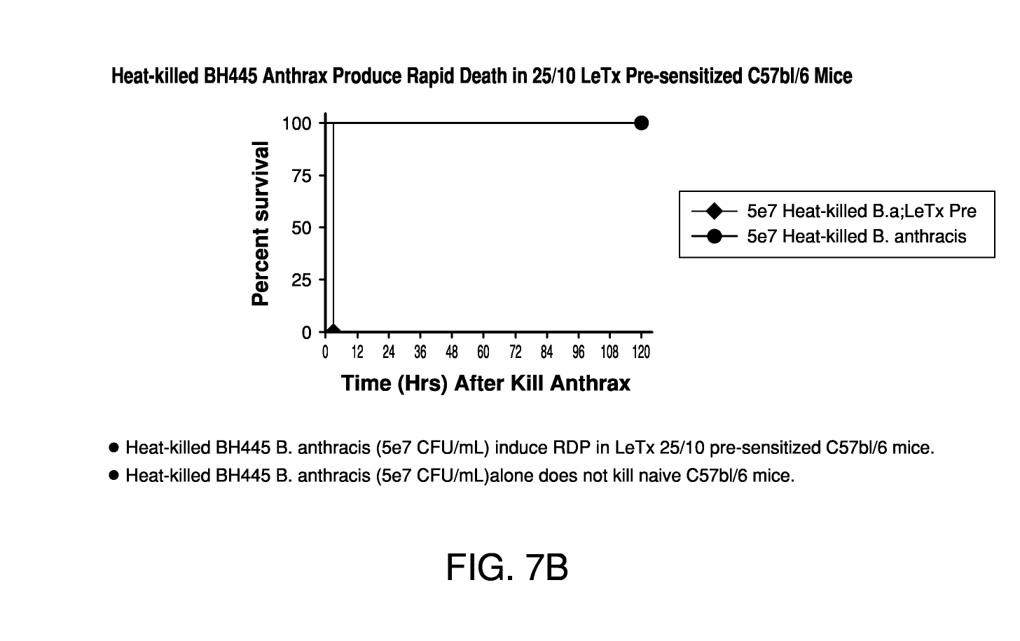

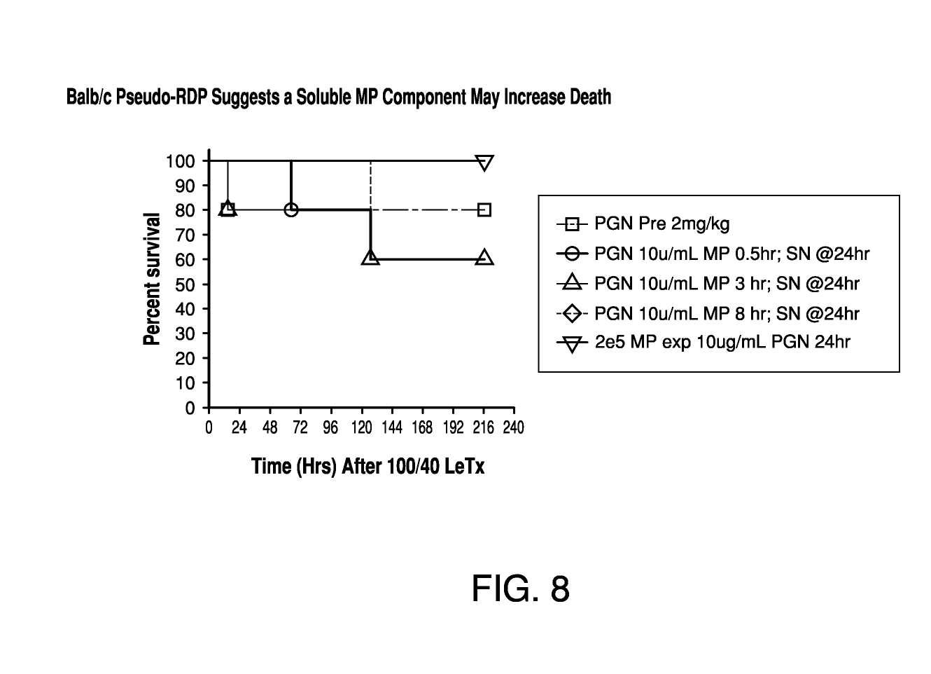

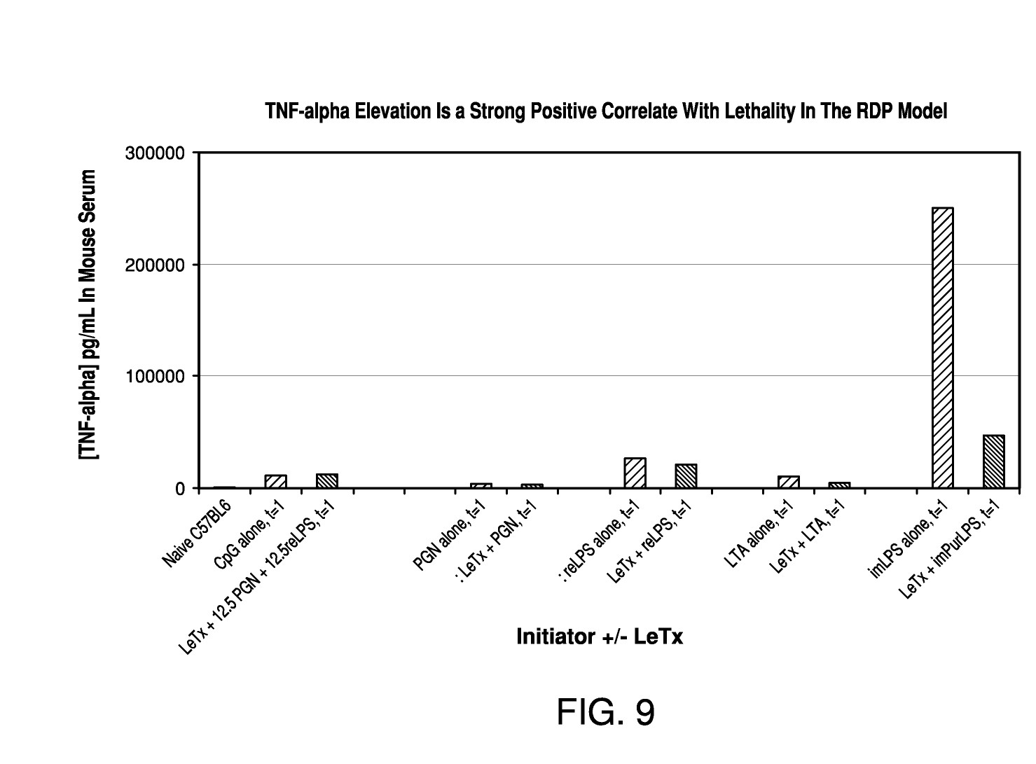

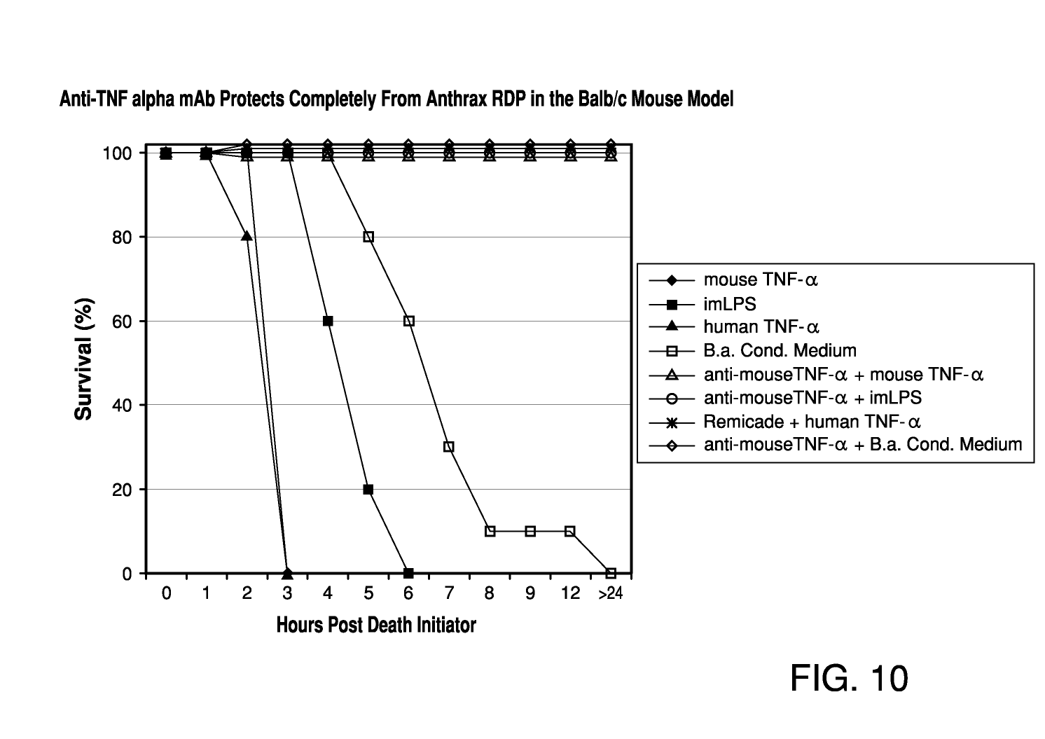

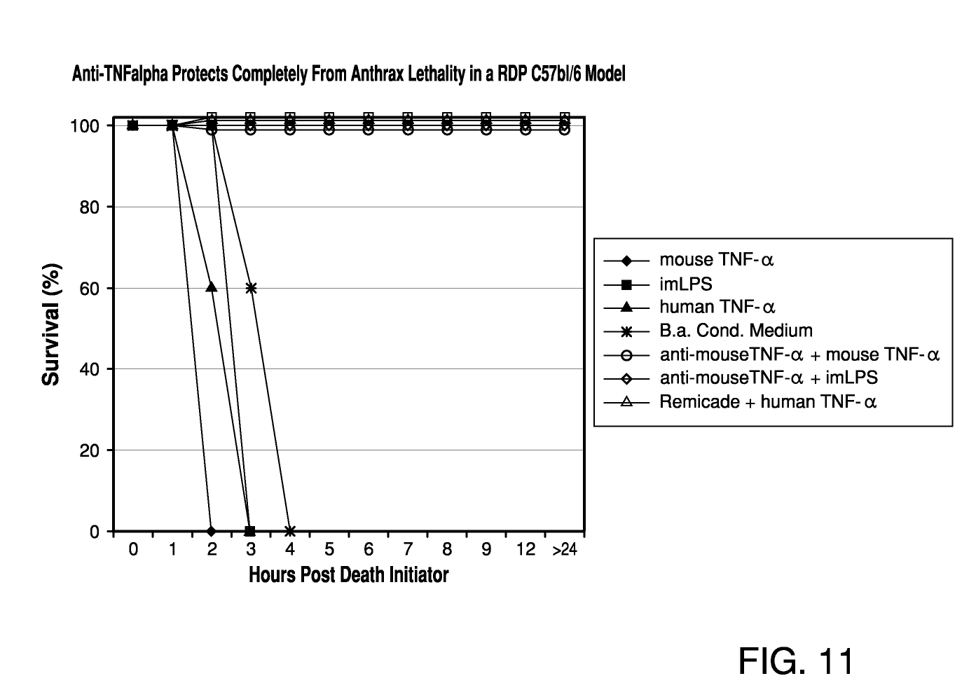

The present findings have elucidated the death initiator for anthrax lethal toxin-mediated death, TNF-α and its downstream effects and sequelae, as derived from the B. anthracis organism (or analogs) that agonize PAMPs. It is demonstrated herein the successful interdiction of that death pathway (see FIGS. 7-11) in a wide variety of models. This has revealed a new therapeutic for anthrax fatalities based on products already approved for human use by the FDA.

The key understanding here is that LeTx attenuates but does not eliminate TNF-α production by LeTx poisoned cells or whole organisms such as the mouse model. An initiator, secondary to Toll-like Receptor agonists, triggers the apoptotic death pathway. This pathway is under investigation by our research team and is believed to involve various “death domains”, “apoptosis cascades” and mitochondrial dysfunction and alteration, resulting finally in a massive fulminant liver shutdown which then feeds into systemic shutdown.

The current findings include the following:

1. Death trigger in a mouse model. The death trigger is mouse TNF-α (<=1 μg in a 20 gram mouse). We have shown TLR and Nod1/Nod2 agonists in the RDP model upregulate TNF-α production (Bio-Plex cytokine array). It has been shown herein that mouse TNF-α is a death trigger for RDP whether administered directly or elicited by varying inflammatory stimuli; and conversely, the neutralization of this mouse TNF-α eliminates death from RDP. Furthermore, by using adoptive transfer of human proteins into this mouse model, it has been shown herein that human TNF-α, a selective mouse TNFR1 receptor agonist, is a death trigger in the mouse model of RDP. Similar results have been obtained herein in two widely different mouse strains, BALB/c and C57bl/6. This death initiator identification is in itself a paradigm shifting discovery.

2. The effectiveness of eliminating this trigger in the mouse RDP model in BOTH mouse strains has been demonstrated. First, we initiated RDP using mouse TNF-α (1 μg). Death was 100% (N=14) within 2 hours. Next, we performed the same RDP in the two mouse strains in the presence of rat anti-mouse -TNF-α (150 μg). Survival was 100% (N=10).

Elimination of this TNF-α trigger is not an artifact but generalized. Next, we challenged LeTx pre-administered mice, RDP, again two strains, with excess LPS (27.5 μg Sigma O11:B4 LPS) a known potent inflammatory agent, a known potent TNF-α upregulator and known RDP death initiator. Death in this single test was 100% in 2-5.5 hours (N=10; prior N>70 100% mortality). We again administered rat anti-mouse-TNF-α (150 μg). Survival was 100% (N=10).

Next we challenged LeTx pre-administered mice, RDP, again two strains, with excess BHI Bacillus anthracis (strain BH445 that produces NO LeTx or EdTx) conditioned medium. We again administered rat anti-mouse-TNF-α monoclonal antibody. Survival was 100% (N=10) with antibody treatment and 0% (N=10) without antibody treatment.

It was further demonstrated that the RDP death initiator (bacterial lipoprotein, BLP: Pam-3-CSK-4, synthetic potent analog) working through a pure Toll-like receptor 2 in Balb/c mice is also abrogated by anti-TNF-α treatment (100% survival from RDP when treated with a rat anti-mouse TNF-α monoclonal antibody).

It was further demonstrated that killed B. anthracis (non-toxigenic) at 1E8 killed bacteria per mouse is a potent death initiator and this too is rendered non-lethal by anti-TNF treatment.

3. We have duplicated the above findings in the mouse models of anthrax RDP using human products. We have shown human TNF-α (Chemicon, Inc.), a selective mouse TNFR1 receptor agonist, is the death trigger in the mouse model of RDP.

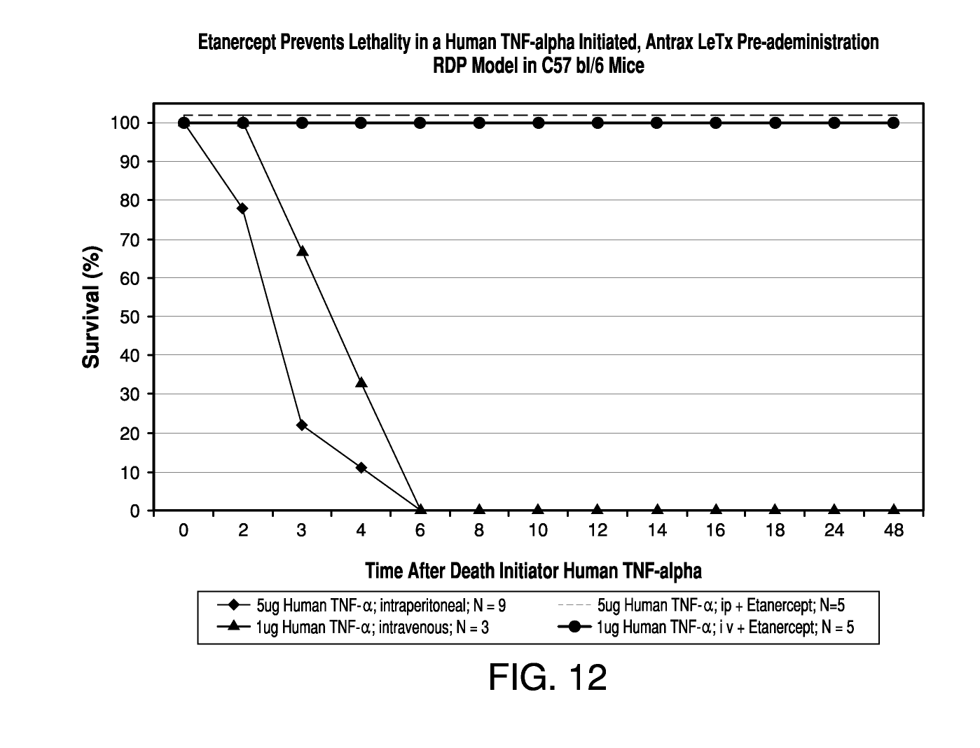

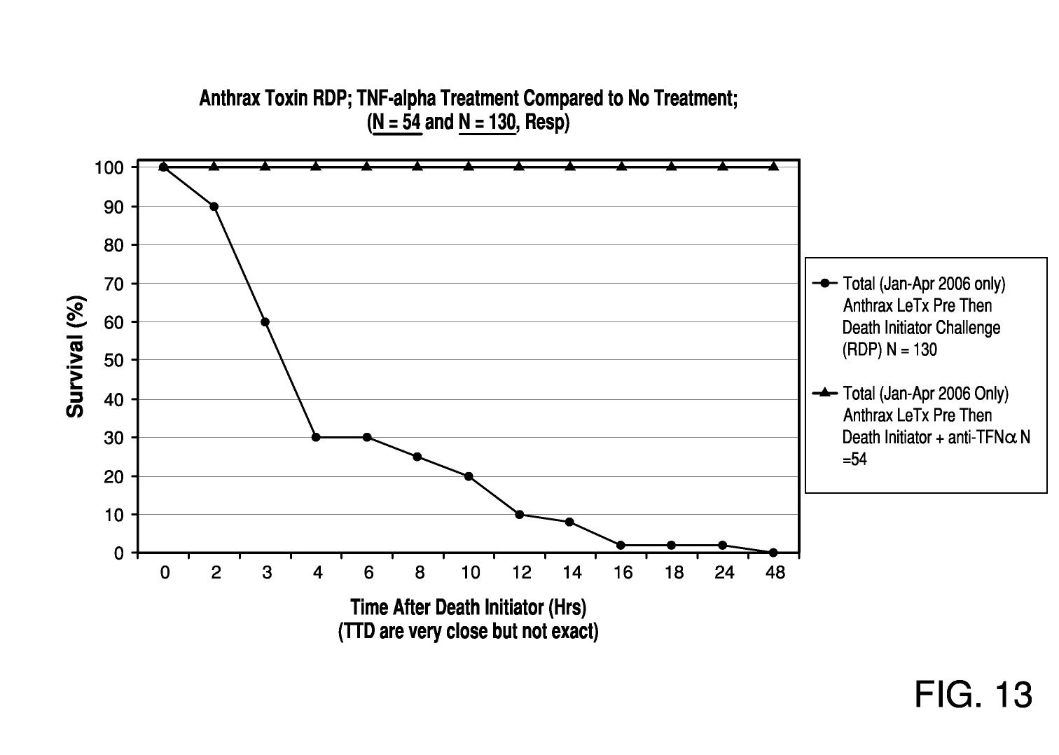

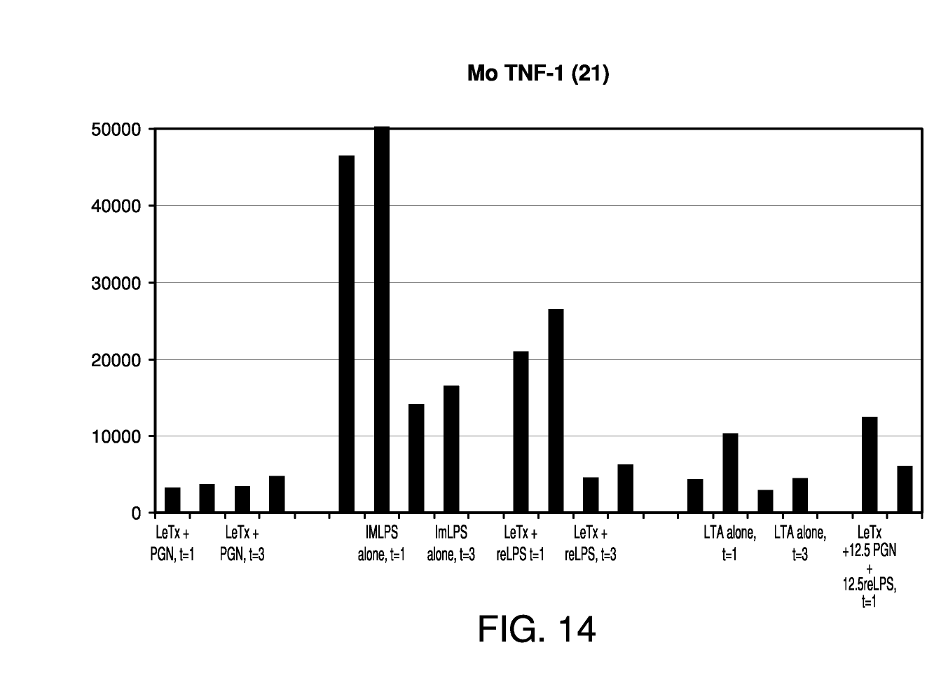

Next, it was shown herein that the effectiveness of eliminating this human TNF-α trigger in the mouse RDP models using an FDA approved anti-human TNF-α monoclonal chimeric humanized antibody known as Infliximab (Remicade), purchased openly at our pharmacy. First, we initiated RDP using human TNF-α (2-5 μg). Death was 100% (N=15) within about 2.0-2.75 hours. Next, we performed the same RDP in the two mouse strains in the presence of chimeric anti-human TNF-α (300 μg) Remicade. Survival was 100% (N=10). It was also shown that (see FIGS. 12-14) Etanercept, a TNFR2 conjugated to an Fc portion of an IgG, protects when the death initiator is human TNF-α

4. The principle of inflammation reduction, sic TNF-α reduction, was further demonstrated by showing that Pirfenidone, an anti-inflammatory that inhibits NFkappaB translocation protein, is impressively protective if given at a specified time delay and that survival enhancement was 40% in one instance and 20% in another.

One hundred thirty mice from two widely different strains were challenged in the RDP model (small, non-lethal dose of LeTx first; followed by a death initiator) in during the past three months alone. 128/130 have died. The two that survived were from lab error; no initiator was administered.

Fifty-nine mice were separately challenged as above, RDP, but with anti-TNF-α treatment. Of these 59 mice, all 59, 100% survived. More negative controls than interventions were used because, not only was the death premise tested, but in addition this premise was tested alongside each and every intervention rather than extrapolating from past experiments.

The invention should not be construed to be limited solely to the assays and methods described herein, but should be construed to include other methods and assays as well. One of skill in the art will know that other assays and methods are available to perform the procedures described herein.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention.

What is claimed is:

1. A method of inhibiting anthrax-associated lethality in a subject in need thereof, said method comprising administering to said subject a pharmaceutical composition comprising an effective amount of at least one TNF-α-inhibiting compound and a pharmaceutically-acceptable carrier.

2. The method of claim 1, wherein the anthrax-associated lethality is regulated by TNF-α.

3. The method of claim 2, wherein the subject is a human.

4. The method of claim 2, wherein the TNF-α-inhibiting compound is selected from the group consisting of a peptide, a protein, a nucleic acid, an antisense oligonucleotide, an siRNA, an aptamer, a kinase inhibitor, and an antibody.

5. The method of claim 2, wherein the TNF-α-inhibiting compound is selected from the group consisting of Remicade, Enbrel, Humira, Segard, Humicade, CDP-571, CDP-870, Onercept, PEG sTNFR1, CytoTab, ISIS-104838, Pirfenidone, BMS561392, Bindarit, GW333, Kineret, and Marimastat.

6. The method of claim 1, wherein said TNF-α-inhibiting compound inhibits TNF-α function.

7. The method of claim 5, wherein said TNF-α-inhibiting compound is an anti-TNF-α antibody.

8. The method of claim 7, wherein said antibody is a monoclonal antibody.

9. The method of claim 7, wherein said antibody is a neutralizing antibody.

10. The method of claim 5, wherein said TNF-α-inhibiting compound is an antibody directed against a protein downstream from TNF-α in its signal transduction pathway.

11. The method of claim 5, wherein said TNF-α-inhibiting compound inhibits cell apoptosis.

12. The method of claim 8, wherein said cell apoptosis is regulated by I-FLICE.

13. The method of claim 1, wherein TNF-α is inhibited by regulating upstream proteins involved in the TNF-α protein regulatory pathway.

14. The method of claim 13, wherein the upstream protein is selected from the group consisting of PI3K, SOCS, and IRAK-M.

15. The method of claim 1, wherein said subject is further administered an effective amount of an enhancer of the innate immune system, and optionally an effective amount of at least one antibiotic.

16. The method of claim 15, wherein said antibiotic is administered before said TNF-α inhibiting compound or said enhancer.

17. The method of claim 15, wherein said subject is further administered an effective amount of a presensitizing agent.

18. The method of claim 17, wherein said antibiotic is administered before said TNF-α inhibiting compound, said enhancer, or said presensitizing agent.

19. The method of claim 15, wherein said enhancer is selected from the group consisting of LPS, ultra pure LPS, PAM-3-CSK-4, PGN, BLP, MDP, Lipid A, dsRNA, poly I:C, ssRNA, CpG DNA, flagellin, and LTA.

20. The method of claim 15, wherein said enhancer of the innate immune system is administered before said TNFα inhibiting compound is administered.

21. The method of claim 15, wherein the innate immune system is enhanced via a Toll-Like receptor.

22. A method of enhancing survival against a pathogen or toxin in a subject in need thereof, said method comprising administering to said subject a pharmaceutical composition comprising an effective amount of an enhancer of the innate immune system, thereby enhancing survival of said subject.

23. The method of claim 22, wherein the pathogen is selected from the group consisting of bacteria, a virus, and a parasite.

24. The method of claim 23, wherein said pathogen is anthrax.

25. The method of claim 24, wherein said subject is a human.

26. The method of claim 22, wherein the enhancer is administered before the subject is contacted with the pathogen or toxin.

27. The method of claim 22, wherein said enhancer is selected from the group consisting of LPS, ultra pure LPS, PAM-3-CSK-4, PGN, BLP, MDP, Lipid A, dsRNA, poly I:C, ssRNA, CpG DNA, flagellin, and LTA.

28. The method of claim 22, wherein said subject is further administered an effective amount of a TNF-α inhibiting compound and optionally an effective amount of an antibiotic or antiviral.

29. A method of protecting a subject from anthrax-associated lethality, said method comprising presensitizing said subject by administering a pharmaceutical composition comprising an effective amount of at least one presensitizing agent prior to infection of said subject with anthrax.

30. The method of claim 29, wherein said subject is further administered an effective amount of at least one antibiotic or antiviral and optionally an effective amount of at least one TNF-α inhibiting compound.

31. A kit for treating anthrax-associated lethality in a subject in need thereof, said kit comprising a pharmaceutical composition comprising an effective amount of at least one TNF-α inhibiting compound packaged in one or more unit dosages, an instructional material for the use thereof, an applicator, and optionally one or more of at least one enhancer of the innate immune system packaged in one or more unit dosages, at least one compound to presensitize the subject to the lethal toxin effects of anthrax packaged in one or more unit dosages, and at least one antibiotic packaged in one or more unit dosages.
